(19) United States
(12) Patent Application Publication
(10) Pub. No.: US 2006/0281088 A1
(43) Pub. Date: Dec. 14, 2006

(54) Markers for lxr activation

(76) Inventor: Matthew Blake Wright, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 10/557,720

(22) PCT Filed: May 14, 2004
(86) PCT No.: PCT/EP04/05217

§ 371 Date: Nov. 18, 2005

(30) Foreign Application Priority Data
May 21, 2003 (EP) 03011091.0

(51) Int. Cl. C12Q 1/68; C07H 21/04

(52) U.S. Cl. 435/6; 536/23.5

(57) ABSTRACT

The present invention relates to surrogate markers for LXR activation, and methods of diagnosing a disease linked to LXR activation, methods of monitoring the treatment of patients suffering from a disease linked to LXR activation, and methods of identifying compounds which modulate LXR activity.

Liver-X-Receptors (LXR) are nuclear hormone receptors that regulate the expression of genes involved in cholesterol and lipid metabolism and bile acid synthesis. LXRs have been implicated in a number of diseases, such as atherosclerosis, dyslipidemia and diabetes. Recent data also suggests a role in inflammation. Several genes have been shown to be regulated by LXR, including ABCA1 (Costet, P., et al., Sterol-dependent transactivation of the ABC1 promoter by the liver X receptor/retinoid X receptor. J Biol Chem, 2000.275(36): p. 28240-5; Schwartz, K., R. M. Lawn, and D. P. Wade, ABC1 gene expression and ApoA-I-mediated cholesterol efflux are regulated by LXR. Biochem Biophys Res Commun, 2000. 274(3): p. 794-802); WO02/070011 discloses ABCA-1 as a surrogate marker for PPAR activation; ABCG1 (Kennedy, M. A., et al., Characterization of the human ABCG1 Gene; LXR activates an internal promoter that produces a novel transcript encoding an alternative form of the protein. J Biol Chem, 2001. 10: p. 10.); ApoC1 (Stulnig, T. M., et al., Novel roles of liver X receptors exposed by gene expression profiling in liver and adipose tissue. Mol Pharmacol, 2002.62(6): p. 1299-305); ApoE (Laffitte, B. A., et al., LXRs control lipid-inducible expression of the apolipoprotein E gene in macrophages and adipocytes. Proc Natl Acad Sci USA, 2001. 98(2): p. 507-12); FAS (Joseph, S. B., et al., Direct and indirect mechanisms for regulation of fatty acid synthase gene expression by LXRs. J Biol Chem, 2002. 14: p. 14); LDLR (Stulnig, T. M., et al., Novel roles of liver X receptors exposed by gene expression profiling in liver and adipose tissue. Mol Pharmacol, 2002. 62(6): p. 1299-305); NR1H3; (Laffitte, B. A., et al., Autoregulation of the Human Liver X Receptor alpha Promoter. Mol Cell Biol, 2001. 21(22): p. 7558-68); SREBPF1 (DeBose-Boyd, R. A., et al., Expression of sterol regulatory element-binding protein 1c (SREBP-1c) mRNA in rat hepatoma cells requires endogenous LXR ligands. Proc Natl Acad Sci USA, 2001. 98(4):p. 1477-82). Also of interest is US2004/0023276, which discloses LXR-ligand induced genes and proteins.

The present invention relates to surrogate markers for LXR activation, and methods of diagnosing a disease linked to LXR activation, methods of monitoring the treatment of patients suffering from a disease linked to LXR activation, and methods of identifying compounds which modulate LXR activity.

The present invention provides a marker for detecting or monitoring LXR modulation, comprising at least one nucleic acid selected from the group consisting of the nucleic acids listed in tables 2 and/or 3. The term “modulation” as used herein relates to an activation or inhibition of the transcriptional activity of LXR. Thus, said nucleic acids can serve as surrogate markers for modulation of LXR activity.

The term “marker” as used herein refers to a single nucleic acid or polypeptide, or a panel of multiple nucleic acids or polypeptides.

Preferably, said nucleic acids are nucleic acids listed in table 2. In a more preferred embodiment, the marker comprises at least one nucleic acid selected from the group consisting of Seq ID No. 1, 4 to 7, 12, 14, 20 to 22 and 26. In another preferred embodiment, said nucleic acids are nucleic acids listed in table 2, with the exception of Seq. ID No. 3, 4, 5 and 20. In another more preferred embodiment, the marker comprises at least one nucleic acid selected from the group consisting of Seq ID No. 1, 6, 7, 12, 14, 21, 22 and 26. In an even more preferred embodiment, the marker comprises at least one nucleic acid selected from the group consisting of Seq ID No. 1, 12 or 21. In a most preferred embodiment, the marker comprises at least one nucleic acid of Seq ID No. 1 or 21. In another embodiment, the marker more preferably comprises at least one nucleic acid selected from the group consisting of the nucleic acids of Seq ID No. 20, 22 or 26, more preferably at least one nucleic acid selected from the group consisting of the nucleic acids of Seq ID No. 22 or 26 and most preferably, the marker comprises the nucleic acid of Seq ID No. 26.

In another preferred embodiment, the marker comprises at least one nucleic acid listed in table 3. More preferably, said at least one nucleic acid is selected from the group consisting of the nucleic acids of Seq ID No. 16, 17 and 23 to 25. In a most preferred embodiment, said at least one nucleic acid is selected from the group consisting of Seq ID No. 16 or 24.

The present invention also pertains to a marker for diagnosing a disease involving dysregulation of LXR activity, comprising one or more of the nucleic acids selected from the group consisting of the nucleic acids listed in tables 2 and/or 3. Thus, said nucleic acids can serve as surrogate markers for the modulation of LXR activity.

Preferably, said nucleic acids are nucleic acids listed in table 2. In a more preferred embodiment, the marker comprises at least one nucleic acid selected from the group consisting of Seq ID No. 1, 4 to 7, 12, 14, 20 to 22 and 26. In another preferred embodiment, said nucleic acids are nucleic acids listed in table 2, with the exception of Seq. ID No. 3, 4, 5 and 20. In another more preferred embodiment, the marker comprises at least one nucleic acid selected from the group consisting of Seq ID No. 16, 7, 12, 14, 21, 22 and 26. In an even more preferred embodiment, the marker comprises at least one nucleic acid selected from the group consisting of Seq ID No. 1, 12 or 21. In a most preferred embodiment, the marker comprises at least one nucleic acid of Seq ID No. 1 or 21. In another embodiment, the marker more preferably comprises at least one nucleic 15 acid selected from the group consisting of the nucleic acids of Seq ID No. 20, 22 or 26, more preferably at least one nucleic acid selected from the group consisting of the nucleic acids of Seq ID No. 22 or 26 and most preferably, the marker comprises the nucleic acid of Seq ID No. 26.

In another preferred embodiment, said at least one nucleic acids are selected from the group consisting of the nucleic acids listed in table 3. More preferably, said at least one nucleic acids are selected from the group consisting of the nucleic acids of Seq ID No. 16, 17 and 23 to 25. Most preferably, said at least one nucleic acids are selected from the group consisting of Seq ID No. 16 and/or 24.

The invention also pertains to a marker for diagnosing a disease involving dysregulation of LXR activity, comprising at least one nucleic acid selected from the group consisting of the nucleic acids listed in table 3 and one or more nucleic acids listed selected from the group consisting of the nucleic acids in table 2. Preferably, the marker comprises one nucleic acid listed in table 3 and one nucleic acid listed in table 2.

The polypeptides encoded by the nucleic acids listed in tables 2 and/or 3 can also be used as markers. Thus, the present invention also provides a marker for detecting or monitoring LXR modulation, comprising one or more polypeptides selected from the group consisting of the polypeptides encoded by the nucleic acids listed in tables 2 and/or 3.

Preferably, said polypeptides are selected from the group consisting of the polypeptides encoded by the nucleic acids listed in table 2. In a more preferred embodiment, the marker comprises at least one polypeptide selected from the group consisting of the polypeptides encoded by the nucleic acids of Seq ID No. 1, 4 to 7, 12, 14, 20 to 22 and 26. In another preferred embodiment, said polypeptides are the polypeptides encoded by the nucleic acids listed in table 2, with the exception of Seq. ID No. 3, 4, 5 and 20. In another more preferred embodiment, the marker comprises at least one polypeptide selected from the group consisting of the polypeptides encoded by the nucleic acids of Seq ID No. 1, 6, 7, 12, 14, 21, 22 and 26. In an even more preferred embodiment, the marker comprises at least one polypeptide encoded by the nucleic acids selected from the group consisting of Seq ID No. 1, 12 or 21. In a most preferred embodiment, the marker comprises at least one polypeptide encoded by the nucleic acids of Seq ID No. 1 or 21. In another embodiment, the marker more preferably comprises at least one polypeptide selected from the group consisting of the polypeptides encoded by the nucleic acids of Seq ID No. 20, 22 or 26, more preferably at least one polypeptide selected from the group consisting of the polypeptides encoded by the nucleic acids of Seq ID No. 22 or 26 and most preferably, the marker comprises the polypeptides encoded by the nucleic acid of Seq ID No. 26.

In another preferred embodiment, said polypeptides are selected from the group consisting of the polypeptides encoded by the nucleic acids listed in table 3. More preferably, said polypeptides are selected from the group consisting of the polypeptides encoded by the nucleic acids of Seq ID No. 16, 17 and 23 to 25. Most preferably, said polypeptides are selected from the group consisting of the polypeptides encoded by the nucleic acids of Seq ID No. 16 and/or 24.

In another embodiment, the present invention provides a marker for detecting or monitoring LXR modulation, comprising at least one polypeptide selected from the group consisting of the polypeptides encoded by the nucleic acid listed in table 3 and one or more polypeptides selected from the group consisting of the polypeptides encoded by the nucleic acids listed in table 2. Preferably, the marker comprises one polypeptide selected from the group consisting of the polypeptides encoded by the nucleic acids listed in table 3 and one polypeptide selected from the group consisting of the polypeptides encoded by the nucleic acids listed in table 2. More preferably, the marker comprises two polypeptides selected from the group consisting of the polypeptides encoded by the nucleic acids listed in table 3 and two polypeptides selected from the group consisting of the polypeptides encoded by the nucleic acids listed in table 2. Even more preferably, the marker comprises three polypeptides selected from the group consisting of the polypeptides encoded by the nucleic acids listed in table 3 and three polypeptides selected from the group consisting of the polypeptides encoded by the nucleic acids listed in table 2. Most preferably, the marker comprises four polypeptides selected from the group consisting of the polypeptides encoded by the nucleic acids listed in table 3 and four polypeptides selected from the group consisting of the polypeptides encoded by the nucleic acids listed in table 2.

Further to this, the present invention also provides a marker for diagnosing a disease involving dysregulation of LXR activity, comprising one or more polypeptides selected from the group consisting of the polypeptides encoded by the nucleic acids listed in tables 2 and/or 3.

Preferably, said polypeptides are selected from the group consisting of the polypeptides encoded by the nucleic acids listed in table 2. In a more preferred embodiment, the marker comprises at least one polypeptide selected from the group consisting of the polypeptides encoded by the nucleic acids of Seq ID No. 1, 4 to 7, 12, 14, 20 to 22 and 26. In another preferred embodiment, said polypeptides are the polypeptides encoded by the nucleic acids listed in table 2, with the exception of Seq. ID No. 3, 4, 5 and 20. In another more preferred embodiment, the marker comprises at least one polypeptide selected from the group consisting of the polypeptides encoded by the nucleic acids of Seq ID No. 1, 6, 7, 12, 14, 21, 22 and 26. In an even more preferred embodiment, the marker comprises at least one polypeptide encoded by the nucleic acids selected from the group consisting of Seq ID No. 1, 12 or 21. In a most preferred embodiment, the marker comprises at least one polypeptide encoded by the nucleic acids of Seq ID No. 1 or 21. In another embodiment, the marker more preferably comprises at least one polypeptide selected from the group consisting of the polypeptides encoded by the nucleic acids of Seq ID No. 20, 22 or 26, more preferably at least one polypeptide selected from the group consisting of the polypeptides encoded by the nucleic acids of Seq ID No. 22 or 26 and most preferably, the marker comprises the polypeptides encoded by the nucleic acid of Seq ID No. 26.

In another preferred embodiment, said polypeptides are selected from the group consisting of the polypeptides encoded by the nucleic acids listed in table 3. More preferably, said polypeptides are selected from the group consisting of the polypeptides encoded by the nucleic acids of Seq ID No. 16, 17 and 23 to 25. Most preferably, said said polypeptides are selected from the group consisting of the polypeptides encoded by the nucleic acids of Seq ID No. 16 and/or 24.

The invention also pertains to a marker for diagnosing a disease involving dysregulation of LXR activity, comprising at least one polypeptide selected from the group consisting of the polypeptides encoded by a nucleic acid listed in table 3 and one or more polypeptides selected from the group consisting of the polypeptides encoded by the nucleic acids listed in table 2. Preferably, the marker comprises one polypeptide selected from the group consisting of the polypeptides encoded by a nucleic acid listed in table 3 and one polypeptide selected from the group consisting of the polypeptides encoded by a nucleic acid listed in table 2. More preferably, the marker comprises at least two polypeptides selected from the group consisting of the polypeptides encoded by a nucleic acid listed in table 3 and at least two polypeptides selected from the group consisting of the polypeptides encoded by a nucleic acid listed in table 2. Even more preferably, the marker comprises at least three polypeptides selected from the group consisting of the polypeptides encoded by a nucleic acid listed in table 3 and at least three polypeptides selected from the group consisting of the polypeptides encoded by a nucleic acid listed in table 2. Most preferably, the marker comprises at least four polypeptides selected from the group consisting of the polypeptides encoded by a nucleic acid listed in table 3 and at least four polypeptides selected from the group consisting of the polypeptides encoded by a nucleic acid listed in table 2.

The nucleic acids and polypeptides which constitute the novel markers hereinbefore described are useful for several processes. A method for screening compounds that modulate LXR activity is provided, comprising the steps of contacting said compounds with a host, and measuring the expression of at least one nucleic acid selected from the group consisting of the nucleic acid listed in tables 2 and/or 3.

Preferably, said nucleic acids are nucleic acids listed in table 2. In a more preferred embodiment, the marker comprises at least one nucleic acid selected from the group consisting of Seq ID No. 1, 4 to 7, 12, 14, 20 to 22 and 26. In another preferred embodiment, said nucleic acids are nucleic acids listed in table 2, with the exception of Seq. ID No. 3, 4, 5 and 20. In another more preferred embodiment, the marker comprises at least one nucleic acid selected from the group consisting of Seq ID No. 1, 6, 7, 12, 14, 21, 22 and 26. In an even more preferred embodiment, the marker comprises at least one nucleic acid selected from the group consisting of Seq ID No. 1, 12 or 21. In a most preferred embodiment, the marker comprises at least one nucleic acid of Seq ID No. 1 or 21. In another embodiment, the marker more preferably comprises at least one nucleic acid selected from the group constisting of the nucleic acids of Seq ID No. 20, 22 or 26, more preferably at least one nucleic acid selected from the group constisting of the nucleic acids of Seq ID No. 22 or 26 and most preferably, the marker comprises the nucleic acid of Seq ID No. 26.

In one embodiment of the method hereinbefore described, the expression of at least one nucleic acid selected from the group consisting of the nucleic acids herein before described is compared to the expression of said at least one nucleic acid in a control. The control can either be an untreated host, which may be the same host before the treatment or a different host, and/or the same host after an appropriate period of treatment for normalization to pretreatment levels. In a preferred embodiment of the method hereinbefore described, the compound that modulates LXR activity is either an antagonist or an agonist.

In another preferred embodiment, said at least one nucleic acids are selected from the group consisting of the nucleic acids listed in table 3. More preferably, said at least one nucleic acids are selected from the group consisting of the nucleic acids of Seq ID No. 16, 17 and 23 to 25. Most preferably, said at least one nucleic acids are selected from the group consisting of Seq ID No. 16 and/or 24.

Several methods for measuring expression of said nucleic acids can be used. Methods such as Northern Blotting, and quantitation of the bands by densitometry are well known in the art and may be used, although they may not be sufficiently accurate. Other methods include the use of genechips, microarray analysis, dot blotting or different quantitative PCR methodologies. Preferably, Taqman or real time quantitative PCR is used. In a preferred embodiment, expression levels of at least one nucleic acids selected from the group consisting of Seq ID No. 16 to 26 are determined by Taqman quantitative PCR using the forward primers, reverse primers and probes listed in table 1. In a more preferred embodiment, the expression levels of at least one nucleic acid selected from the group consisting of Seq ID No. 20, 21, 22 and 26 are measured by Taqman quantitative PCR. In a most preferred embodiment, the primers and probes used for measuring at least one nucleic acid selected from the group consisting of Seq ID No. 20, 21, 22 and 26 are primers and protes of Seq ID No. 39 to 41 (for measuring expression levels of the nucleic acid of Seq ID No. 20), or Seq ID No. 42 to 44 (for measuring expression levels of the nucleic acid of Seq ID No. 21), or Seq ID No. 45 to 47 (for measuring expression levels of the nucleic acid of Seq ID No. 22), or Seq ID No. 57 to 59 (for measuring expression levels of the nucleic acid of Seq ID No. 26).

Taqman quantitative PCR is performed as follows:

Three oligonucleotides are used: a forward primer, a reverse primer, and a probe. All of them are specific for the target and are able to bind to it. The TaqMan assay uses a probe technology that exploits 5′→3′-nuclease activity of an enzyme, the most commonly used being Taq polymerase. The assay efficiency is largely dependent on this 5′→3′ nuclease activity. In this regard one should be careful in choosing a suitable polymerase. Indeed, some polymerases available on the market appear not to be suitable for real-time RT-PCR, even though the manufacturers claim they possess 5′-exonuclease activity. The probe is an oligonucleotide with a reporter dye at the 5′ end and a quencher dye at the 3′ end. The fluorescent reporter dye is attached covalently to the 5′ end and can be FAM (6-carboxyfluorescein), TET (tetrachloro-6-carboxyfluorescein), JOE (2,7-dimethoxy-4,5-dichloro-6-carboxyfluorescein), HEX (hexacholoro-6-carboxyfluorescein), or VIC. The reporter is quenched by TAMRA (6-carboxytetramethylrhodamine), bound to the 3′ end by a linker arm. DABCYL [4-(48-dimethylaminophenylazo)benzoic acid] can also be used as a quencher dye, but its use is much more prevalent in the molecular beacon probes. An advantage of using DABCYL in the TaqMan probes is its reduced autofluorescence compared with TAMRA When the probe is intact the quencher dye absorbs the fluorescence of the reporter dye due to the proximity between both. The proximity between quencher and fluorophore permits FRET, and fluorescence emission does not occur. By the 5′-exonuclease activity of the Taq:polymerase the probe is hydrolyzed and the reporter dye is separated from the quencher, resulting in an increase in fluorescence emission. During PCR amplification, if the target of interest is present, the probe specifically anneals to the target. The Taq polymerase cleaves the probe, allowing an increase in fluorescence emission. This increase in fluorescence is measured cycle by cycle and is a direct consequence of the amplification process (Giulietti, A., et al., An Overview of eal-Time Quantitative PCR: Applications to Quantify Cytokine Gene Expression. Methods 25, 386-401 (2001)).

Another method provided by the present invention is a method for screening compounds that modulate LXR activity, comprising the steps of contacting said compounds with a host, and measuring the expression of at least one polypeptide selected from the group consisting of the polypeptides encoded by the nucleic acids listed in tables 2 and/or 3. Preferably, said polypeptides are selected from the group consisting of the polypeptides encoded by the nucleic acids listed in table 2. In a more preferred embodiment, the marker comprises the nucleic acids of, or the polypeptides encoded by the nucleic acids of Seq ID No. 1, 4 to 7, 12, 14, 20 to 22 and 26. In another preferred embodiment, the marker comprises the nucleic acids, or the polypeptides encoded by the nucleic acids listed in table 2, with the exception of Seq ID No. 3 to 5 and 20. In another more preferred embodiment, the marker comprises the nucleic acids of, or the polypeptides encoded by the nucleic acids of Seq ID No. 1, 6, 7, 12, 14, 21, 22 and 26. In an even more preferred embodiment, the marker comprises the nucleic acids of, or the polypeptides encoded by the nucleic acids of Seq ID No. 1, 12 or 21. In a most preferred embodiment, the marker comprises the nucleic acids of, or the polypeptides encoded by the nucleic acids of Seq ID No. 1 or 21. In another embodiment, the marker more preferably comprises the nucleic acids of, or the polypeptides encoded by the nucleic acids of Seq ID No. 20, 22 or 26, and most preferably, the marker comprises the nucleic acid of, or the polypeptide encoded by Seq ID No. 26.

In another preferred embodiment, said polypeptides are selected from the group consisting of the polypeptides encoded by the nucleic acids listed in table 3. More preferably, said polypeptides are selected from the group consisting of the polypeptides encoded by the nucleic acids of Seq ID No. 16, 17 and 23 to 25. Most preferably, said polypeptides are selected from the group consisting of the polypeptides encoded by the nucleic acids of Seq ID No. 16 and/or 24.

In another embodiment of the method hereinbefore described, the expression of at least one of the polypeptides is compared to the expression of said at least one polypeptide in a control. The control can be an untreated host or a host treated with a carrier. The carrier may be the solvent in which the compound is dissolved or resuspended. In a preferred embodiment of the method hereinbefore described, the compound that modulates LXR activity is either an antagonist or an agonist.

The present invention also provides a method for monitoring treatment of patients suffering from a disease associated with dysregulation of LXR activity, comprising the steps of purifying mRNA or protein from monocytes/macrophages or from total blood isolated from patients treated with a modulator of LXR activity and measuring the expression of at least one of the nucleic acids, or at least one of the polypeptides encoded by one of the nucleic adds listed in tables 2 and/or 3.

Preferably, said polypeptides are encoded by the nucleic acids listed in table 2. In a more preferred embodiment, the marker comprises the nucleic acids of, or the polypeptides encoded by the nucleic acids of Seq ID No. 1, 4 to 7, 12, 14, 20 to 22 and 26. In another preferred embodiment, the marker comprises the nucleic acids, or the polypeptides encoded by the nucleic acids listed in table 2, with the exception of Seq ID No. 3 to 5 and 20. In another more preferred embodiment, the marker comprises the nucleic acids of, or the polypeptides encoded by the nucleic acids of Seq ID No. 1, 6, 7, 12, 14, 21, 22 and 26. In an even more preferred embodiment, the marker comprises the nucleic acids of, or the polypeptides encoded by the nucleic acids of Seq ID No. 1, 12 or 21. In a most preferred embodiment, the marker comprises the nucleic acids of, or the polypeptides encoded by the nucleic acids of Seq ID No. 1 or 21. In another embodiment, the marker more preferably comprises the nucleic acids of, or the polypeptides encoded by the nucleic acids of Seq ID No. 20, 22 or 26, and most preferably, the marker comprises the nucleic acid of, or the polypeptide encoded by Seq ID No. 26.

In another preferred embodiment, said polypeptides are selected from the group consisting of the polypeptides encoded by the nucleic acids listed in table 3. More preferably, said polypeptides are selected from the group consisting of the polypeptides encoded by the nucleic acids of Seq ID No. 16, 17 and 23 to 25. Most preferably, said polypeptides are selected from the group consisting of the polypeptides encoded by the nucleic acids of Seq ID No. 16 and/or 24.

The control can either be an untreated host, which may be the same host before the treatment or a different host, and/or the same host after an appropriate period of treatment for normalization to pretreatment levels.

The present invention also provides a method for diagnosing a disease involving dysregulation of LXR activity, comprising the steps of extracting mRNA or protein from total blood or from purified monocytes/macrophages, and measuring the expression of at least one of the nucleic acids, or at least one of the polypeptides encoded by one of the nucleic acids listed in tables 2 or 3.

Preferably, said polypeptides are encoded by the nucleic acids listed in table 2. In a more preferred embodiment, the marker comprises the nucleic acids of, or the polypeptides encoded by the nucleic acids of Seq ID No. 1, 4 to 7, 12, 14, 20 to 22 and 26. In another preferred embodiment, the marker comprises the nucleic acids, or the polypeptides encoded by the nucleic acids listed in table 2, with the exception of Seq ID No. 3 to 5 and 20. In another more preferred embodiment, the marker comprises the nucleic acids of, or the polypeptides encoded by the nucleic acids of Seq ID No. 1, 6, 7, 12, 14, 21, 22 and 26. In an even more preferred embodiment, the marker comprises the nucleic acids of, or the polypeptides encoded by the nucleic acids of Seq ID No. 1, 12 or 21. In a most preferred embodiment, the marker comprises the nucleic acids of, or the polypeptides encoded by the nucleic acids of Seq ID No. 1 or 21. In another embodiment, the marker more preferably comprises the nucleic acids of, or the polypeptides encoded by the nucleic acids of Seq ID No. 20, 22 or 26, and most preferably, the marker comprises the nucleic acid of, or the polypeptide encoded by Seq ID No. 26.

In another preferred embodiment, said at least one nucleic acids or polypeptides are selected from the group consisting of the nucleic acids, or the polypeptides encoded by the nucleic acids, listed in table 3. More preferably, said at least one nucleic acids or polypeptides are selected from the group consisting of the nucleic acids, or the polypeptides encoded by the nucleic acids of Seq ID No. 16, 17 and 23 to 25. Most preferably, said at least one nucleic acids or polypeptides are selected from the group consisting of the nucleic acids, or polypeptides encoded by the nucleic acids of Seq ID No. 16 and/or 24.

The control can either be an untreated host, which may be the same host before the treatment or a different host, and/or the same host after an appropriate period of treatment for normalization to pretreatment levels.

Further to the methods and markers hereinbefore described, the present invention provides a use of one or more nucleic acids, or one or more polypeptides encoded by the nucleic acids listed in tables 2 and/or 3 as a marker for LXR modulation. Preferably, said polypeptides are encoded by the nucleic acids listed in table 2. In a more preferred embodiment, the marker comprises the nucleic, acids of, or the polypeptides encoded by the nucleic acids of Seq ID No. 1, 4 to 7, 12, 14, 20 to 22 and 26. In another preferred embodiment, the marker comprises the nucleic acids, or the polypeptides encoded by the nucleic acids listed in table 2, with the exception of Seq ID No. 3 to 5 and 20. In another more preferred embodiment, the marker comprises the nucleic acids of, or the polypeptides encoded by the nucleic acids of Seq ID No. 1, 6, 7, 12, 14, 21, 22 and 26. In an even more preferred embodiment, the marker comprises the nucleic acids of, or the polypeptides encoded by the nucleic acids of Seq ID No. 1, 12 or 21. In a most preferred embodiment, the marker comprises the nucleic acids of, or the polypeptides encoded by the nucleic acids of Seq ID No. 1 or 21. In another embodiment, the marker more preferably comprises the nucleic acids of, or the polypeptides encoded by the nucleic acids of Seq ID No. 20, 22 or 26, and most preferably, the marker comprises the nucleic acid of, or the polypeptide encoded by Seq ID No. 26.

In another preferred embodiment, said at least one nucleic acids or polypeptides are selected from the group consisting of the nucleic acids, or the polypeptides encoded by the nucleic acids, listed in table 3. More preferably, said at least one nucleic acids or polypeptides are selected from the group consisting of the nucleic acids, or the polypeptides encoded by the nucleic acids of Seq ID No. 16, 17 and 23 to 25. Most preferably, said at least one nucleic acids or polypeptides are selected from the group consisting of the nucleic acids, or polypeptides encoded by the nucleic acids of Seq ID No. 16 and/or 24.

The present invention also pertains to compounds identified by the methods hereinbefore described, and to the use of compounds identified by a method hereinbefore described for the preparation of a medicament for the treatment of a disease involving dysregulation of LXR activity.

### EXAMPLES

The invention will now be illustrated by reference to the following examples which should not be construed as a limitation thereto.

Cell Culture and RNA Preparation

The myelogenous monocytic leukemia cell line (THP1; ATCC TID-202) was cultured in RPMI 1640 medium with Phenol Red containing 2 g/l glucose and 2 mM Glutamine, 10% Fetal Bovine Serum FBS (v/v), (Gibco), 1% Penicillin-Streptomycin P/S (v/v) (Gibco), 50 □M 2-Mercaptoethanol (Bio-Rad) and 1% 100 mM Sodium Pyruvate (v/v) (Gibco). Cells were incubated at 37° C. and 5.2% CO₂. For experimental treatments, cells were seeded at a density of 1×10⁶/ml in 6-well plates in medium containing 100 nM phorbol 12-myristate 13-acetate (PMA) for 72 hours to fully differentiate them into adherent macrophages. The medium was then replaced by culture medium containing PMA with vehicle (DMSO) or test compound for 6 or 24 hours prior to harvesting. Total RNA was isolated using the TriZol reagent (Life Technologies) or the RNeasy mini kit (Qiagen) according to the manufacturer's protocols. To remove contaminating DNA, the samples were treated with RNAse free DNAse (GibCo).

Gene Expression Measurement by DNA Chips

Synthesis of first and second strand cDNA were performed using the SuperScript Choice Gene Chip Kit (Life Technologies) and reagents from Gibco. The double stranded cDNA, containing an incorporated T7 RNA polymerase binding site, was purified by extraction with a mix of phenol:chloroform:isoamylalcohol (Life Technologies). The organic and aqueous phases were separated by Phase Lock Gel (Eppendorf) and double-stranded cDNA was recovered by precipitation according to the manufacturer's protocol and then resuspended in water.

The double-stranded cDNA was converted to biotin-labeled cRNA by in vitro transcription (IVT) using a T7 kit (Ambion) and biotin-containing ribonucleotides (Enzo-LOXO GmbH). The IVT-material was purified from unincorporated ribonucleotides using RNeasy spin columns (Qiagen). Following cleanup, the single-stranded biotin-labeled cRNA were chemically hydrolyzed to smaller fragments in 500 mM calcium acetate, 150 mM magnesium acetate, pH 8.1 for 35 min at 95° C. The reaction was terminated by chilling samples on ice.

Probes were hybridized to the HGU95AV2 A GeneChip Microarray (Affymetrix) which contains features representing ˜12,000 genes. All washing, hybridization, detection, and signal amplification steps were performed using a GeneChip Fluidics Station (Affymetrix). Fluorescence intensity data was collected from the hybridized GeneArrays using a GeneArray scanner (Affymetrix). The raw files containing the fluorescence intensity information were transformed into data files using the Affymetrix Microarray Suite (MAS) software. Differentially expressed genes were identified using the Roche Affymetrix Chip Experiment Analysis (RACE-A) software. Differences between vehicle-treated vs test compound-treated wells (n=4 per group) were evaluated by student t-test and expressed as fold change vs control.

Gene Expression Measurement by Taqman Quantitative PCR

Gene expression levels were measured by Taqman quantitative RT-PCR on an ABI PRISM 7700 sequence detection system (Applied Biosystems). Total RNA was reverse transcribed into cDNA using the Superscript II RT kit (Life Technologies). The primer/probe sequences used are listed in Table I. Primers were designed using PrimerSelect Software (Perkin Elmer). Differences between vehicle-treated vs test compound-treated wells (n=4 per group) were evaluated by student t-test and expressed as fold change vs control. TaqMan quantitative PCR was performed as described (Giulietti, A., et al., An Overview of eal-Time Quantitative PCR: Applications to Quantify Cytokine Gene Expression. Methods 25, 386-401 (2001)).

### Example 1

LXRalpha and LXRbeta are nuclear receptors that heterodimerize with RXR and have been shown to regulate expression of several target genes such as ABCA1, ABCG1, SREBF1, FASN, LPL, and CYP7A [1-9]. To identify surrogate genes that could be measured in patients to evaluate efficacy of a treatment regime, THP1 macrophages were treated with a selective LXR ligand. At 200 nM, T0901317 [10] is a selective LXR agonist (EC50=30 nM and 10 nM on human LXRalpha and LXRbeta, respectively). Gene expression changes were evaluated by Gene Chip Microarray analysis. The LXR agonist T0901317 showed strong induction of ABCA1, ABCG1, APOC1, APOE, ASM3A, C3F, CDC42EP4, CXCR4, PASN, LDLR, NR1H3, SREBP1, VLDLR, FADS1, FADS2, CYB561, IL15, PPFIA2, SERPINI1, KIAA0763, PPIC, LASSI, NFATC4, NR4A3, PRODH2, PRKCI (Table 2). Thus, all of these genes are surrogate markers for LXR modulation and their activity can be measured using GeneChip Microarray technology.

### Example 2

To evaluate an alternative method of measuring surrogate marker genes, THP1 macrophages were treated with T0901317 as described above. Gene expression changes were measured by Taqman quantitative PCR for ABCA1, ABCG1, APOC1, APOE, ASM3A, CDC42EP4, CXCR4, FASN, LDLR, NR1H3, VLDLR. The selective LXR agonist produced a robust and significant increase in expression of each of these marker genes (Table 3). These results, taken together, suggest that measurement of mRNA expression of surrogate marker genes by multiple methods can be effectively used to evaluate therapeutic efficacy of an LXR treatment regimen.

TABLE I
List of Taqman primers and probes used in this study

| | | Seq | | Seq |
|---|---|---|---|---|
| Gene | | ID | | ID |
| Name | Forward Primer | No | Reverse Primer | No. |
| A. | | | | |
| ABCA1 | AACCCACCACAGGCATGG | 27 | ACACTTAGGGCACAATTCCACA | 28 |
| ABCG1 | CCCTCCAGTCATGTTCTTCGA | 30 | ATGATGGAGCGACCCCCT | 31 |
| APOC1 | CATGAGGCTCTTCCTGTCGC | 33 | TGGGCCTTCCAAGACGATC | 34 |
| APOE | CGTTGCTGGTCACATTCCTG | 36 | GCTCTGTCTCCACCGCTTG | 37 |
| ASM3A | GAATCTAAAGGGAGAGTCCATCTGG | 39 | TCCGGCTGCAAATCTTCAAT | 40 |
| CDC42EP | TCGGGTATGAGCCCCTGAG | 42 | GGAGGTGGGTCAGGCTGTT | 43 |
| CXCR4 | GCAGGACCTGTGGCCAAGT | 45 | CGCTCTGGAATGTTCAGTTCC | 46 |
| FASN | TTGCATTGCTGGTAGAGACCC | 48 | CACACGCTGCCTGAGGAGT | 49 |
| LDLR | CCCCAGGGACAAAACACTGT | 51 | GCTCCGAAACCAGAAAGGCT | 52 |
| NR1H3 | TGTAACCGGCGCTCCTTTT | 54 | TGGTGCCATGGGCCA | 55 |
| VLDLR | CAAATAATACCCCCGTCGGA | 57 | CCAGCCGAGAGGAAGAAAAA | 58 |

| | | Seq |
|---|---|---|
| Gene | | ID |
| Name | Probe | No. |
| B. | | |
| ABCA1 | TCCCAAAGCCCGGCGGTTC | 29 |
| ABCG1 | CCCTCCAGTCATGTTCTTCGA | 32 |
| APOC1 | CCCGGTCCTGGTGGTGGTTCTGT | 35 |
| APOE | CAGGATGCCAGGCCAAGGTGGA | 38 |
| ASM3A | AGCTGGAGTATATCCTGACCCAGACCTACGA | 41 |
| CDC42EP | TTGACTGCCGGTTATTTTTCTGTCCTGG | 44 |
| CXCR4 | TTAGTTGCTGTATGTCTCGTGGTAGGACTGTAGAAAA | 47 |
| FASN | CAGGCCTGTCCACCCTGCCAA | 50 |
| LDLR | CCCCCCAGTGCAGGGAACCG | 53 |
| NR1H3 | TGACCGGCTTCGAGTCACGCC | 56 |
| VLDLR | TGGTAACCGAGCCAGCAGCTGAAGTCT | 59 |

All sequences are depicted in the 5′ to 3′ direction. The Taqman probes were labeled with 5′-FAM and 3′-TAMRA and purified by HPLC.

TABLE II
Identification of surrogate marker genes of LXR activation by Gene
Chip Microarrays

| | | | | | Treatment | Seq ID |
|---|---|---|---|---|---|---|
| Gene Name | Unigene ID | Accession | Fold-change | p-Value | Time (hrs) | No |
| ASM3A | Hs.42945 | NM 006714.1 | +2.84 | <0.001** | 6 | 20 |
| C3F | Hs.300423 | NM 005768.3 | +9.5 | 0.038* | 6 | 1 |
| CDC42EP4 | Hs.3903 | AF099664.1 | +39.62 | <0.001** | 24 | 21 |
| CXCR4 | Hs.89414 | NM 003467.1 | +3.05 | <0.001** | 6 | 22 |
| VLDLR | Hs.73729 | D16493.1 | +3.88 | 0.001** | 24 | 26 |
| FADS1 | Hs.132898 | NM 013402.3 | +2.11 | <0.001** | 24 | 3 |
| FADS2 | Hs.184641 | NM 004265.2 | +3.24 | 0.012* | 24 | 4 |
| CYB561 | Hs.355264 | BC002976.1 | +3.16 | 0.002** | 24 | 5 |
| IL15 | Hs.168132 | NM 000585.2 | +3.44 | 0.03* | 24 | 6 |
| PPFIA2 | Hs.30881 | AF034799.1 | +3.1 | 0.016* | 24 | 7 |
| SERPINI1 | Hs.78589 | NM 005025.1 | +2 | <0.001** | 24 | 8 |
| KIAA0763 | Hs.4764 | AB018306.1 | +2.38 | <0.001** | 24 | 9 |
| PPIC | Hs.110364 | NM 000943.2 | +2.99 | 0.001** | 24 | 10 |
| LASS1 | Hs.348258 | NM 021267.1 | +2.26 | <0.001** | 24 | 11 |
| NFATC4 | Hs.77810 | NM 004554.1 | +5.62 | 0.01* | 24 | 12 |
| NR4A3 | Hs.80561 | X89894.1 | +1.99 | <0.001** | 24 | 13 |
| PRODH2 | Hs.128834 | NM 021232.1 | +3.87 | 0.001** | 24 | 14 |
| PRKCI | Hs.1904 | NM002740.1 | +2.28 | <0.001** | 24 | 15 |

Fold change in mRNA levels in THP1 macrophages treated with LXR agonist T0901317 as measured by Gene Chip Microarray Analysis.
All values are fold-change in mRNA and represent the difference between the means of 4 treated vs 4 controls.
Time of treatment is 6 or 24 hours.
*significant p < 0.05;
**highly significant, p < 0.01.

TABLE III
Identification of surrogate marker genes of LXR activation by Gene
Chip Microarrays

| | | | | | Treatment | Seq ID |
|---|---|---|---|---|---|---|
| Gene Name | Unigene ID | Accession | Fold-change | p-Value | Time (hrs) | No |
| ABCA1 | Hs.211562 | AJ012376.1 | +3.95 | <0.001** | 6 | 16 |
| ABCG1 | Hs.10237 | NM 004915.2 | +12.85 | <0.001** | 6 | 17 |
| APOC1 | Hs.268571 | NM 001645.2 | +2.77 | 0.003** | 24 | 18 |
| APOE | Hs.169401 | NM 000041.1 | +1.83 | 0.04* | 24 | 19 |
| FASN | Hs.83190 | NM 004104.3 | +2.09 | 0.009** | 24 | 23 |
| LDLR | Hs.213289 | NM 000527.2 | +1.78 | 0.001** | 24 | 24 |
| NR1H3 | Hs.347353 | NM 005693.1 | +4.1 | 0.001** | 6 | 25 |
| SREBF1 | Hs.166 | NM 004176.2 | +1.92 | 0.001** | 24 | 2 |

Fold change in mRNA levels in THP1 macrophages treated with LXR agonist T0901317 as measured by Gene Chip Microarray Analysis.
All values are fold-change in mRNA and represent the difference between the means of 4 treated vs 4 controls.
Time of treatment is 6 or 24 hours.
*significant p < 0.05;
**highly significant, p < 0.01.

TABLE IV
Evaluation of surrogate marker genes of LXR activation by Taqman
quantitative RT-PCR

| | | Fold- | | Treatment | Seq ID |
|---|---|---|---|---|---|
| Gene Name | Unigene ID | change | p-Value | Time (hrs) | No |
| ABCA1 | Hs.211562 | +9.52 | <0.001** | 6 | 16 |
| ABCG1 | Hs.10237 | +110.02 | <0.001** | 6 | 17 |
| APOC1 | Hs.268571 | +4.31 | <0.001** | 24 | 18 |
| APOE | Hs.169401 | +2.2 | <0.001** | 24 | 19 |
| ASM3A | Hs.42945 | +6.9 | 0.01* | 6 | 20 |
| CDC42EP4 | Hs.3903 | +1.86 | <0.001** | 24 | 21 |
| CXCR4 | Hs.89414 | +3.29 | <0.001** | 6 | 22 |
| FASN | Hs.83190 | +1.42 | 0.02* | 24 | 23 |
| LDLR | Hs.213289 | +2.02 | 0.01* | 24 | 24 |
| NR1H3 | Hs.347353 | +5.02 | 0.001** | 6 | 25 |
| VLDLR | Hs.73729 | +6.63 | 0.001** | 24 | 26 |

Fold change in mRNA levels in THP1 macrophages treated with LXR agonist T0901317 as measured by Taqman Quantitative RT-PCR.
All values are fold-change in mRNA and represent the difference between the means of 4 treated vs 4 controls.
Time of treatment is 6 or 24 hours.
*significant p < 0.05;
**highly significant, p < 0.01.

1-5. (canceled)

6. A method for screening compounds that modulate LXR activity, comprising the steps of contacting said compounds with a host, and measuring the expression of at least one of the nucleic acids listed in tables 2 and/or 3.

7. The method of claim 6, wherein the expression of at least one of the nucleic acids listed in tables 2 and/or 3 is compared to a control.

8. A method for screening compounds that modulate LXR activity, comprising the steps of: contacting said compounds with a host, and measuring the expression of at least one of the polypeptides encoded by the nucleic acids listed in tables 2 and/or 3.

9. The method of claim 8 wherein said nucleic acid or nucleic acids is/are selected from the group consisting of Seq ID No. 1,6,7,12,14,21,22 and 26.

10. The method of claim 9 wherein said nucleic acid or nucleic acids is/are SEQ ID No. 1 and/or 21.

11. The method of claim 8 wherein said nucleic acid or nucleic acids is/are selected from the group consisting of SEQ ID No. 1,4 to 7, 12, 14, 20 to 22 and 26.

12. (canceled)

13. A method for monitoring treatment of patients suffering from a disease associated with dysregulation of LXR activity, comprising the steps of:
purifying mRNA or protein from monocytes/macrophages or from total blood isolated from patients treated with a modulator of LXR activity and measuring the expression of at least one of the nucleic acids, or at least one of the polypeptides encoded by one of the nucleic acids listed in table 2 and/or 3.

14. The method of claim 13, wherein the expression of at least one of the nucleic acids, or at least one protein encoded by the nucleic acids listed in tables 2 and/or 3 is compared to a control.

15. A method for diagnosing a disease involving dysregulation of LXR activity, comprising the steps of extracting mRNA from total blood or from purified monocytes/macrophages, and measuring the expression of at lease one of the nucleic acids, or at least one of the polypeptides encoded by one of the nucleic acids listed in tables 2 and/or 3.

16. The method of claim 15, wherein the expression of at least one of the nucleic acids, or at least one protein encoded by the nucleic acids listed in tables 2 and/or 3 is compared to a control.

17-19. (canceled)


SEQUENCE LISTING (59 sequences)

SEQ ID NO: 1 - DNA, 2058 bp, Homo sapiens
  Homo sapiens putative protein similar to nessy (Drosophila) (C3F) (1)..(2058): representative cDNA sequence (NM_005768.3, as of 24 March 2003) for gene under Unigene ID Hs.300423
ggcacgagggttacccctttgctttgttttatcggcattaccttttctacaaggagacctacctcatccacctcttccatacctttacaggcctctcaattgcttattttaactttggaaaccagctctaccactccctgctgtgtattgtgcttcagttcctcatccttcgactaatgggccgcaccatcactgccgtcctcactaccttttgcttccagatggcctaccttctggctggatactattacactgccaccggcaactacgatatcaagtggacaatgccacattgtgttctgactttgaagctgattggtttggctgttgactactttgacggagggaaagatcagaattccttgtcctctgagcaacagaaatatgccatacgtggtgttccttccctgctggaagttgctggtttctcctacttctatggggccttcttggtagggccccagttctcaatgaatcactacatgaagctggtgcagggagagctgattgacataccaggaaagataccaaacagcatcattcctgctctcaagcgcctgagtctgggccttttctacctagtgggctacacactgctcagcccccacatcacagaagactatctcctcactgaagactatgacaaccaccccttctggttccgctgcatgtacatgctgatctggggcaagtttgtgctgtacaaatatgtcacctgttggctggtcacagaaggagtatgcattttgacgggcctgggcttcaatggctttgaagaaaagggcaaggcaaagtgggatgcctgtgccaacatgaaggtgtggctctttgaaacaaacccccgcttcactggcaccattgcctcattcaacatcaacaccaacgcctgggtggcccgctacatcttcaaacgactcaagttccttggaaataaagaactctctcagggtctctcgttgctattcctggccctctggcacggcctgcactcaggatacctggtctgcttccagatgaaattcctcattgttattgtggaaagacaggctgccaggctcattcaagagagccccaccctgagcaagctggccgccattactgtcctccagcccttctactatttggtgcaacagaccatccactggctcttcatgggttactccatgactgccttctgcctcttcacgtgggacaaatggcttaaggtgtataaatccatctatttccttggccacatcttcttcctgagcctactattcatattgccttatattcacaaagcaatggtgccaaggaaagagaagttaaagaagatggaataatccatttccctggtggcctgtgcgggactggtgcagaaactactcgtctcccttttcacagcactcctttgccccagagcagagaatggaaaagccagggaggtggaagatcgatgcttccagctgtgcctctgctgccagccaagtcttcatttggggccaaaggggaaacttttttttggagaaggcgtcttgctttgtcacccacgctggaatgcagtggcgggatctcagctcaccgcaacctccacctcctgggttcaagtgattttcctgcctcagcctcccaagtagctgggaatacaggcacgccaccatgcccagctaatttttgtattttcagtagaaacgggatttcaccacgttggccaggctggtctcgaactcctgaccgcaagtgatccacccgcctccgcctcccaaagtgctgggattacaggcgtgagccaccgtgcccggcccaaaggggaaactcttgtgggaggagcagaggggctcacatctcccctctgattcccccatgcacattgccttatctctccccatctagccaggaatctattgtgtttttcttctgccaatttactatgattgtgtatgtgccgctaccaccaccccccccatgggggggtggagaggggtgcaaggccctgcctgctccactttttctaccttggaactgtattagataaaatcacttctgtttgttcagtttttcaaaaaaaaaaaaaaaaaa

SEQ ID NO: 2 - DNA, 4154 bp, Homo sapiens
  Homo sapiens sterol regulatory element binding transcription factor 1 (SREBF1) (1)..(4154): representative cDNA sequence (NM_004176.2, as of 24 March 2003) for gene under Unigene ID Hs.Hs.166
taacgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttata

SEQ ID NO: 3 - DNA, 4213 bp, Homo sapiens
  Homo sapiens fatty acid desaturase 1 (FADS1) (1)..(4213): representative cDNA sequence (NM_013402.3, as of 24 March 2003) for gene under Unigene ID Hs.132898
tccactcctggagcccgcggaccccgagcacgcgcctgacagcccctgctggcccggcgcgcggcgtcgccaggccagctatggcccccgacccggtggccgccgagaccgcggctcagggacctaccccgcgctacttcacctgggacgaggtggcccagcgctcagggtgcgaggagcggtggctagtgatcgaccgtaaggtgtacaacatcagcgagttcacccgccggcatccagggggctcccgggtcatcagccactacgccgggcaggatgccacggatccctttgtggccttccacatcaacaagggccttgtgaagaagtatatgaactctctcctgattggagaactgtctccagagcagcccagctttgagcccaccaagaataaagagctgacagatgagttccgggagctgcgggccacagtggagcggatggggctcatgaaggccaaccatgtcttcttcctgctgtacctgctgcacatcttgctgctggatggtgcagcctggctcaccctttgggtctttgggacgtcctttttgcccttcctcctctgtgcggtgctgctcagtgcagttcaggcccaggctggctggctgcagcatgactttgggcacctgtcggtcttcagcacctcaaagtggaaccatctgctacatcattttgtgattggccacctgaagggggcccccgccagttggtggaaccacatgcacttccagcaccatgccaagcccaactgcttccgcaaagacccagacatcaacatgcatcccttcttctttgccttggggaagatcctctctgtggagcttgggaaacagaagaaaaaatatatgccgtacaaccaccagcacaaatacttcttcctaattgggcccccagccttgctgcctctctacttccagtggtatattttctattttgttatccagcgaaagaagtgggtggacttggcctggatgattaccttctacgtccgcttcttcctcacttatgtgccactattggggctgaaagccttcctgggccttttcttcatagtcaggttcctggaaagcaactggtttgtgtgggtgacacagatgaaccatattcccatgcacattgatcatgaccggaacatggactgggtttccacccagctccaggccacatgcaatgtccacaagtctgccttcaatgactggttcagtggacacctcaacttccagattgagcaccatctttttcccacgatgcctcgacacaattaccacaaagtggctcccctggtgcagtccttgtgtgccaagcatggcatagagtaccagtccaagcccctgctgtcagccttcgccgacatcatccactcactaaaggagtcagggcagctctggctagatgcctatcttcaccaataacaacagccaccctgcccagtctggaagaagaggaggaagactctggagccaaggcagaggggagcttgagggacaatgccactatagtttaatactcagagggggttgggtttggggacataaagcctctgactcaaactcctcccttttatcttctagccacagttctaagacccaaagtggggggtggacacagaagtccctaggagggaaggagctgttggggcaggggtgtaaattatttcctttttctagtttggcacatgcaggtagttggtgaacagagagaaccaggagggtaacagaagaggagggacctactgaacccagagtcaggaagagatttaacactaaaattccactcatgccgggcgtggtggcacgcgcctgtaatcccagctacccaggaggctgaggcaggagaatcgcttgaaccggggaggtggaggttgcagtgagctgagatcacgccattgtactccagcctgggcgacagagcaagactccatttcaaaaaaaaaaaaaaaatccactcatataaaaggtgagctcagctcactggtccatttctcagtggcttctccatcctcatttgcaaacctcagagggataaggcagttgaacctgatgagcaagaattataacagcaaggaaacattaatgcttagaattctgagatccagcacaactcagtctgtgggagctcagctcgctgcccagggataggtatgacctatgtctgccttaggctgctgggagatgccattctccagtttcagaagcaggcagggcaaaggtcaagactgtggtattggggtcttttggctctgaaggatcctggaaccactgattttggtttattccctccagggtctaaagagaacaagaggtgctagctcttaccaaaacagatggtagagagagttgctggctatttaaaaagctctttcatcttttaattcacctcttcttttcacctctttaaccactcctcaggaacagaacacttctaggactgggggtcttttagctccataagcaagtgagcagatgggacaagttagtcttttctccctagaaacaaaggggatgcccagtggtttccctttgcttcccaacctaaaatttcaagtttaataaaatagcaattagcagaagtgaccaaattgggagataattatcagtcatgaggaaagacacagatttcggtcataaagaatgtaagggctataagtagaaactttctataacctaaatgatgttatagaattatttttgagcaggagcagaaagattaaatatgatcacttcatacttctaaatcagaaataggaagattaaaaccacagaacagtttgtgatttctattgctgtagctaggtatcttactctgtccactcttgttcaagtatctaactcttctggaaaccaaataggctttagaagagattatcctatattcctatcagtataatactaaaatgtaactttttaatcatctggtttttaaaagataaacagtttagcccatctctccagagagcaaacataggaatatgactcaggagcctcctagggcttatcatcagccctcacacccgcttccccctccaacccacagcctttgcttccaggtggcaggattactactttgcctcttcagcagcatctactctaggcatattgatcattttagacactgggagaagagaacctcaaactaggaggaaaagacagagcctccacttagttttgggaggggatggcagacagtcaaggagatgagcgtcctaaggcatgttgggatagggtcagatgcaccacccatggagaggtttgtcaacacaaagacatggaaggttagaggtttgtcaacaaaaagacatggaaggttaggtttgtcaacacaaagacatggaagattagaggtttgtcaacacaaagacacaggaagaatgggctgcagaagatttagatgttttccatttgggcacattttacttagctggagaactaggtttaaaacagcctgggtaggaaaattagaagcaagctggatgcagtggctcatgcctgtaatcccaacacttttgggaggtccaggcaggaggatcacttgggcccaggaggtcaagcctgcagcgagctgagatcacaccactgcactccagcctggggtgatagaacaagaccctgtctcaaaaaaaaaaaaaaacaacaaaaacttagaattgaggagttgtacctccattggcttcctcactccaaaataggtgctgatccttcctattcctattctttgccaccttttgggtgtggtgtcaccagcctgtttagccaagtagctttgggcataggctgcccaatctgagcaaacaccagtgaggctctattgagccaagaccaagtcctcaaagcacctgaaccactgtggccttctcagcctacagcagtgtggtctcttacatggccacaaagggacacacagtgacaaaaggctcggaatgttacaatggtaaaatgagtgatctcaaatccactgacagatataaaataggcttagagaggaaaagctgcctctggtcaagtagatcatggcagcatgaattccaactcacttttttacaactccaacttctatgtttatctttgttactttcacttttttacaacctggccagaggcattttttaaatcaggcccaatatcagtattctttttgtgtgtgccaattttgttatcacatccctatgaagttgaaaaataaagttaattttgaccaaaaaaaaaaaaaaaag

SEQ ID NO: 4 - DNA, 3149 bp, Homo sapiens
  Homo sapiens fatty acid desaturase 2 (FADS2) (1)..(3149): representative cDNA sequence (NM_004265.2, as of 24 March 2003) for gene under Unigene ID Hs.184641
agggggcgcggtgggaggagtaggagaagacaaaagccgaaagcgaagagggcccgggctgcacacaccggctgggaggcagccgtctgtgcagcgagcagccggcgcggggaggccgcagtgcacggggcgtcacagtcggcaggcagcatggggaagggagggaaccagggcgagggggccgccgagcgcgaggtgtcggtgcccaccttcagctgggaggagattcagaagcataacctgcgcaccgacaggtggctggtcattgaccgcaaggtttacaacatcaccaaatggtccatccagcacccggggggccagcgggtcatcgggcactacgctggagaagatgcaacggatgccttccgcgccttccaccctgacctggaattcgtgggcaagttcttgaaacccctgctgattggtgaactggccccggaggagcccagccaggaccacggcaagaactcaaagatcactgaggacttccgggccctgaggaagacggctgaggacatgaacctgttcaagaccaaccacgtgttcttcctcctcctcctggcccacatcatcgccctggagagcattgcatggttcactgtcttttactttggcaatggctggattcctaccctcatcacggcctttgtccttgctacctctcaggcccaagctggatggctgcaacatgattatggccacctgtctgtctacagaaaacccaagtggaaccaccttgtccacaaattcgtcattggccacttaaagggtgcctctgccaactggtggaatcatcgccacttccagcaccacgccaagcctaacatcttccacaaggatcccgatgtgaacatgctgcacgtgtttgttctgggcgaatggcagcccatcgagtacggcaagaagaagctgaaatacctgccctacaatcaccagcacgaatacttcttcctgattgggccgccgctgctcatccccatgtatttccagtaccagatcatcatgaccatgatcgtccataagaactgggtggacctggcctgggccgtcagctactacatccggttcttcatcacctacatccctttctacggcatcctgggagccctccttttcctcaacttcatcaggttcctggagagccactggtttgtgtgggtcacacagatgaatcacatcgtcatggagattgaccaggaggcctaccgtgactggttcagtagccagctgacagccacctgcaacgtggagcagtccttcttcaacgactggttcagtggacaccttaacttccagattgagcaccacctcttccccaccatgccccggcacaacttacacaagatcgccccgctggtgaagtctctatgtgccaagcatggcattgaataccaggagaagccgctactgagggccctgctggacatcatcaggtccctgaagaagtctgggaagctgtggctggacgcctaccttcacaaatgaagccacagcccccgggacaccgtggggaaggggtgcaggtggggtgatggccagaggaatgatgggcttttgttctgaggggtgtccgagaggctggtgtatgcactgctcacggaccccatgttggatctttctccctttctcctctcctttttctcttcacatctcccccatagcaccctgccctcatgggacctgccctccctcagccgtcagccatcagccatggccctcccagtgcctcctagccccttcttccaaggagcagagaggtggccaccgggggtggctctgtcctacctccactctctgcccctaaagatgggaggagaccagcggtccatgggtctggcctgtgagtctccccttgcagcctggtcactaggcatcacccccgctttggttcttcagatgctcttggggttcataggggcaggtcctagtcgggcagggcccctgaccctcccggcctggcttcactctccctgacggctgccattggtccaccctttcatagagaggcctgctttgttacaaagctcgggtctccctcctgcagctcggttaagtacccgaggcctctcttaagatgtccagggccccaggcccgcgggcacagccagcccaaaccttgggccctggaagagtcctccaccccatcactagagtgctctgaccctgggctttcacgggccccattccaccgcctccccaacttgagcctgtgaccttgggaccaaagggggagtccctcgtctcttgtgactcagcagaggcagtggccacgttcagggaggggccggctggcctggaggctcagcccaccctccagcttttcctcagggtgtcctgaggtccaagattctggagcaatctgacccttctccaaaggctctgttatcagctgggcagtgccagccaatccctggccatttggccccaggggacgtgggccctgcaggctgcaggagggcactggagctgggaggtctcgtcccagccctccccatctcggggctgctgtgtggacggcgctgcctcaggcactctcctgtctgaacctgcccttactgtgtttaacctgttgctccaggatgcattctgataggagggggcggcagggctgggccttgtgacaatctgcctttcaccacatggccttgcctcggtggccctgactgtcagggagggccagggaggcagagcgggagggagtctcaggaggaggctgccctgaggggctggggagggggtacctcatgaggaccagggtggagctgagaagaggaggaggtgggggctggaggtgctggtagctgaggggacgggcaagtgagaggggagggagggaagtcctgggaggatcctgagctgctgttgcagtctaacccactaatcagttcttagattcaggggaagggcaggcaccaacaactcagaatgggggctttcggggagggcgcctagtccccccagctctaagcagccaggagggacctgcatctaagcatctgggttgccatggcaatggcatgccccccagctactgtatgcccccgacccccgcagaggcagaatgaacccatagggagctgatcgtaatgtttatcatgttacttccccacccctacattttttgaaataaaataaggaattttattctc

SEQ ID NO: 5 - DNA, 2194 bp, Homo sapiens
  Homo sapiens, Similar to cytochrome b-561, clone MGC2190 (1)..(2194): representative cDNA sequence (BC002976.1, as of 24 March 2003)  for gene under Unigene ID Hs.3555264
cctcgtgccgagcgacccgggcaagcgacccgggcgcggcgcggggaggctgaagggacgctcgggtaggcaagcgtttgcctcagcatggagggcggggcctcggcagccacccccacagcactgccttactacgtggccttctcccagctgctgggcctgaccttggtggccatgaccggcgcgtggctcgggctgtaccgaggcggcattgcctgggagagcgacctgcagttcaacgcgcaccccctctgcatggtcataggcctgatcttcctgcagggaaatgccctgctggtttaccgtgtcttcaggaacgaagctaaacgcaccaccaaggtcctgcacgggctgctgcacatctttgcgctcgtcatcgccctggttggcttggtggcggtgttcgactaccacaggaagaagggctacgctgacctgtacagcctacacagctggtgcgggatccttgtctttgtcctgtactttgtgcagtggctggtgggcttcagcttcttcctgttccccggagcttcattctccctgcggagccgctaccgcccacagcacatcttctttggtgctaccatcttcctcctttccgtgggcaccgccctgctgggcctgaaggaggcactgctgttcaacctcgggggcaagtatagcgcatttgagcccgagggtgtcctggccaacgtgctgggcctgctgctggcctgcttcggtggggcggtgctctacatcttgacccgggccgactggaagcggccttcccaggcggaagagcaggccctctccatggacttcaagacgctgacggagggagatagccccggctcccagtgatgcgcccggccggccctgggggttcgcggggtgtcttcttgcctgcccctgctgaggcgtcttcaggactgcaggctccggagagtggctctggcagcaggcgggcgcgtgggtgcagctgcatctgtttgagtgctgctttctggggtcaggtctccgcctcctctgcttctcctttctccgctgctatagaccagttcattgtgtgtggctcccgtgtctctgttgcccccttcagtgcagaaggctttgggtaggacttcgggtgttcggtcctggtcgcagagcacagatctttaaagaagcgagagaggaggccccaccctcctggcagcagatgcctggggcaaggccaggggaaactgggggggcctcagggacaggcctggaaaggccacgatggctgctgaattcaaacaaggagtccctccagcctgaataacacgtggcacaaatgggcccggcctttggcagaggagcaagtgatatgatgtgtaaagtatgttggtggtgaaagcaaggttccccaggagaggggagggactggcccctgggaagctctgagatgaggctgtggcccagctgtagtcctgaccttcctcttctttaaccctttagccctaggatggctttggtgggagaggggatagaagcccatgacttcagacagactttctcttggcagatgcaggcgggcctcctcccaggctgctccagacatgggggttggggatggggggcaccttgcagccccttcctgctggggctccctccttgtagcaccccccttgcggctcagctctggtttcctctcccaggctcacccaggctctgctcaggctgggaggcagagggcacaaaccttataattttttaaatgaaaaaccgctgctgctggctgtggctagagccccctggggctgctggagctgctgcctctgttctggaggacgagccttctccttatctgctgcccatctttccaggaagtcaggatggagtcagaacaactacagtcatcccccgtggtgtctgcacatcactccagccccataaagagtgtcatgttagctgagtcaccatttggcttcggcctggaaatagtgtgattagaacactgatcgtgtgcgaggccaggagatcaagaccatcctgactaacaaacacagtgaaaccccgtctctactaaaaatacaaaaaaattagccaggcgtggtggtgggcgcctgtagtcccagctacttgggaggctgaggcaggagaatggtgtgaacccgggagatggcgcttgcagtgagctgagattgcactccagcctgggcgacaggctcaaaaaaaaaaaaaaaaaaaa

SEQ ID NO: 6 - DNA, 1496 bp, Homo sapiens
  Homo sapiens interleukin 15 (IL15), transcript variant 3 (1)..(1496): representative cDNA sequence (NM_000585.2, as of 24 March 2003) for gene under Unigene ID Hs.168132
gactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgctgacgtcacatggagcacagaaatcaatgttagcagatagccagcccatacaagatcgtattgtattgtaggaggcatcgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcaaaaaaaaaaaaaaaaaaaaaaaaa

SEQ ID NO: 7 - DNA, 4060 bp, Homo sapiens
  Homo sapiens liprin-alpha2 mRNA, PPFIA2 (1)..(4060)
gattccgggaggcaagtgaggagagaagatgctgtagcgtcctcaccggctgccagcagggaaatggtccaggagtgctgggtgtgagcctcccttctcctcaagccggagactgcggttgtcattgatcaattgaagaagcaaggacccgaaatcacagacattagcaatgatgtgtgaagtgatgcccacgattaatgaggacaccccaatgagccaaagggggtcccaaagcagtggctcggactcagactcccattttgagcagctgatggtgaatatgctagatgaaagggatcgtcttctagacacccttcgggagacccaggaaagcctctcacttgcccagcaaagacttcaggatgtcatctatgaccgagactcactccagagacagctcaattcagccctgccacaggatatcgaatccctaacaggagggctggctggttctaagggggctgatccaccggaatttgctgcactgacaaaagaattaaatgcctgcagggaacaacttctagaaaaggaagaagaaatctctgaacttaaagctgaaagaaacaacacaagactattactggagcatttggagtgccttgtgtcacgacatgaaagatcactaagaatgacggtggtaaaacggcaagcccagtctccctcaggagtatccagtgaagttgaagttctcaaggcactgaaatctttgtttgagcaccacaaggccttggatgaaaaggtaagggagcgactgagggtttctttagaaagagtctctgcactggaagaagaactagctgctgctaatcaggagattgttgccttgcgtgaacaaaatgttcatatacaaagaaaaatggcatcaagcgagggatccacagagtcagaacatcttgaagggatggaacctggacagaaagtccatgagaagcgtttgtccaatggttctatagactcaaccgatgaaactagtcaaatagttgaactacaagaattgcttgaaaagcaaaactatgaaatggcccagatgaaagaacgtttagcagccctttcttcccgagtgggagaggtggaacaggaagcagagacagcaagaaaggatctcattaaaacagaagaaatgaacaccaagtatcaaagggacattagggaggccatggcacaaaaggaagatatggaagaaagaattacaacccttgaaaagcgttacctcagtgctcagagagaatctacctccatacatgacatgaatgataaactagaaaatgagttagcaaataaagaagctatcctacggcagatggaagagaaaaacagacagttacaagaacgtcttgagctagctgaagaaaagttgcagcagaccatgagaaaggctgaaaccttgcctgaagtagaggctgaactggctcagagaattgcagccctaaccaaggctgaagagacacatggaaatattgaagaacgtatgagacatttagagggtcaacttgaagagaagaatcaagaacttcaaagagctaggcaaagagagaaaatgaatgaggagcataacaagagattatcggatacggttgatagacttctgactgaatccaatgaacgcctacaactacacttaaaggaaagaatggctgctctagaagaaaagaatgttttaattcaagaatcagaaactttcagaaagaatcttgaagaatctttacatgataaggaaagcttagcagaagaaattgaaaagctgagatctgaacttgaccaattgaaaatgagaactggctctttaattgaacccacaataccaagaactcatctagacacctcagctgagttgcggtactcagtgggatccctagtggacagccagtctgattacagaacaactaaagtaataagaagaccaaggagaggccgcatgggtgtgcgaagagatgagccaaaggtgaaatctcttggggatcacgagtggaatagaactcaacagattggagtactaagcagccacccttttgaaagtgacactgaaatgtctgatattgatgatgatgacagagaaacaatttttagctcaatggatcttctctctccaagtggtcattccgatgcccagacgctagccatgatgcttcaggaacaattggatgccatcaacaaagaaatcaggctaattcaggaagaaaaagaatctacagagttgcgtgctgaagaaattgaaaatagagtggctagtgtgagcctcgaaggcctgaatttggcaatggtccacccaggtacctccattactgcctctgttacagcttcatcgctggccagttcatctccccccagtggacactcaactccaaagctcacccctcgaagccctgccagggaaatggatcggatgggagtcatgacactgccaagtgatctgaggaaacatcggagaaagattgcagttgtggaagaagatggtcgagaggacaaagcaacaattaaatgtgaaacttctcctcctcctacccctagagccctcagaatgactcacactctcccttcttcctaccacaatgatgctcgaagtagtttatctgtctctcttgagccagaaagcctcgggcttggtagtgccaacagcagccaagactctcttcacaaagcccccaagaagaaaggaatcaagtcttcaataggacgtttgtttggtaaaaaagaaaaagctcgacttgggcagctccgaggctttatggagactgaagctgcagctcaggagtccctggggttaggcaaactcggaactcaagctgagaaggatcgaagactaaagaaaaagcatgaacttcttgaagaagctcggagaaagggattaccttttgcccagtgggatgggccaactgtggtcgcatggctagagctttggttgggaatgcctgcgtggtacgtggcagcctgccgagccaacgtgaagagtggtgccatcatgtctgctttatctgacactgagatccagagagaaattggaatcagcaatccactgcatcgcttaaaacttcgattagcaatccaggagatggtttccctaacaagtccttcagctcctccaacatctcgaactccttcaggcaacgtttgggtgactcatgaagaaatggaaaatcttgcagctccagcaaaaacgaaagaatctgaggaaggaagctgggcccagtgtccggtttttctacagaccctggcttatggagatatgaatcatgagtggattggaaatgaatggcttcccagcttggggttacctcagtacagaagttactttatggaatgcttggtagatgcaagaatgttagatcacctaacaaaaaaagatctccgtgtccatttaaaaatggtggatagtttccatcgaacaagtttacaatatggaattatgtgcttaaagaggttgaattatgacagaaaagaactagaaagaagacgggaagcaagccaacatgaaataaaagacgtgttggtgtggagcaatgaccgagttattcgctggatacaagcaattggacttcgagaatatgcaaataatatacttgagagcggtgtgcatggctcacttatagccctggatgaaaactttgactacagcagcttagctttattattacagattccaacacagaacacccaggcaaggcagattcttgaaagagaatacaataacctcttggccctgggaactgaaaggcgactggatgaaagtgatgacaagaacttcagacgtggatcaacctggagaaggcagtttcctcctcgtgaagtacatggaatcagcatgatgcctgggtcctcagaaacattaccagctggatttaggttaaccacaacctctgggcagtcaagaaaaatgacaacagatgttgcttcatcaagactgcagaggttagacaactccactgttcgcacatactcatgttgaccagccactcaaaggaggcagcactgacctgctatggcgtcttttcagtctactctacctaaagtgcactaccatctaagaagacgagcagtgaaaacctttgtgaaaactgaattc

SEQ ID NO: 8 - DNA, 1559 bp, Homo sapiens
  Homo sapiens serine (or cysteine) proteinase inhibitor, clade Ineuroserpin), member 1 (SERPINI1) (1)..(1559): representative cDNA sequence (NM_005025.1, as of 24 March 2003) for gene under Unigene ID Hs.78589
gcggagcacagtccgccgagcacaagctccagcatcccgtcaggggttgcaggtgtgtgggaggcttgaaactgttacaatatggctttccttggactcttctctttgctggttctgcaaagtatggctacaggggccactttccctgaggaagccattgctgacttgtcagtgaatatgtataatcgtcttagagccactggtgaagatgaaaatattctcttctctccattgagtattgctcttgcaatgggaatgatggaacttggggcccaaggatctacccagaaagaaatccgccactcaatgggatatgacagcctaaaaaatggtgaagaattttctttcttgaaggagttttcaaacatggtaactgctaaagagagccaatatgtgatgaaaattgccaattccttgtttgtgcaaaatggatttcatgtcaatgaggagtttttgcaaatgatgaaaaaatattttaatgcagcagtaaatcatgtggacttcagtcaaaatgtagccgtggccaactacatcaataagtgggtggagaataacacaaacaatctggtgaaagatttggtatccccaagggattttgatgctgccacttatctggccctcattaatgctgtctatttcaaggggaactggaagtcgcagtttaggcctgaaaatactagaaccttttctttcactaaagatgatgaaagtgaagtccaaattccaatgatgtatcagcaaggagaattttattatggggaatttagtgatggctccaatgaagctggtggtatctaccaagtcctagaaataccatatgaaggagatgaaataagcatgatgctggtgctgtccagacaggaagttcctcttgctactctggagccattagtcaaagcacagctggttgaagaatgggcaaactctgtgaagaagcaaaaagtagaagtatacctgcccaggttcacagtggaacaggaaattgatttaaaagatgttttgaaggctcttggaataactgaaattttcatcaaagatgcaaatttgacaggcctctctgataataaggagatttttctttccaaagcaattcacaagtccttcctagaggttaatgaagaaggctcagaagctgctgctgtctcaggaatgattgcaattagtaggatggctgtgctgtatcctcaagttattgtcgaccatccatttttctttcttatcagaaacaggagaactggtacaattctattcatgggacgagtcatgcatcctgaaacaatgaacacaagtggacatgatttcgaagaactttaagttactttatttgaataacaaggaaaacagtaactaagcacattatgtttgcaactggtatatatttaggatttgtgttttacagtatatcttaagataatatttaaaatagttccagataaaaacaatatatgtaaattataagtaacttgtcaaggaatgttatcagtattaagctaatggtcctgttatgtcattgtgtttgtgtgctgttgtttaaaataaaagtacctattgaacatgtg

SEQ ID NO: 9 - DNA, 4148 bp, Homo sapiens
  Homo sapiens mRNA for KIAA0763 protein (1)..(4148): representative cDNA sequence (AB018306.1, as of 24 March 2003)  for gene under Unigene ID Hs.4764
gacctccatcctgcgcaagcaggctgaggaggaggccatcaagcgctcacgctcactctccgagagctatgagctctcctcggacctgcaggacaagtaggtggagatgctagaacgaaagtatggggggcgcctggtaacccgccatgcggcccgcaccatccagacggcgtttcgccagtaccagatgaacaagaacttcgagcgcttgcgcagctccatgtcagagaaccgcatgtcacgccggattgtgctgtccaacatgaggatgcagttctcctttgaggggcctgagaaagtgcacagctcctacttcgaggggaagcaggtctcagtgactaacgacggctcccagctgggagccctggtgtcccctgagtgtggtgacctcagcgagcccaccaccctcaagtctccggccccctccagtgactttgcggacgccatcaccgagctggaggacgccttctctaggcaagtgaaatcactggccgagtccatcgacgatgccctcaactgccgcagcctgcacactgaggaggcaccggccctggatgcggcgcgggcccgggacaccgaaccccagacagccctgcacggcatggaccaccgcaaactggacgagatgacggcctcgtacagtgatgtcaccctgtacatcgatgaggaggagctgtcgccccctctgcccctctcgcaggcaggggaccggccgtccagcaccgagtcggacctgcggctacgggctgggggcgcagccccagactactgggccctggcccacaaagaggacaaggctgacacggacacgagctgccggagcacgccgtcgctggagcggcaggagcagcggctgcgggtggagcatctgccgctgctcaccatcgagccacccagcgacagctctgtggaccttagtgaccgctcggagcgggggtcactcaagaggcagagtgcttacgagcgcagccttggcgggcagcagggcagtcccaagcatggtccccacagcggcgcccccaagagcctcccccgggaggagcctgagttgcggccccggccccccaggcccctggacagccacttggccatcaatggctcagccaaccggcagagcaagtctgagtcggactactcagacggtgacaatgacagcatcaacagcacgtccaactccaacgataccatcaactgcagctccgagtcatcgtcccgtgacagcctgcgggagcagacgctcagcaagcagacctaccacaaggaggcccgcaacagctgggactcgcctgcctttagcaacgatgtcatccgcaagaggcactaccgcatcggcctgaacctcttcaacaagaagcctgagaagggagtccagtacctcatcgagcgtggctttgtgcccgacacgcccgtcggggtggcccacttcctgctgcagcgcaagggcctcagccggcagatgatcggcgagttcctgggcaaccggcagaagcagttcaaccgtgacgtgctcgactgcgtcgtggacgagatggacttctctaccatggagctggatgaggccctcaggaaattccaggcgcacatccgtgtccaaggggaggctcagaaagtggagcggctcatagaggcgttcagccagcgctactgcatctgcaaccctggggtggtgcggcaattccggaacccagacaccattttcatcctggccttcgccatcatcctgctgaacaccgacatgtacagccccaatgtcaagcccgagcggaaaatgaagctagaggacttcatcaagaacctccgaggtgtggacgatggtgaggacattccccgtgagatgctgatggggatctatgaacggatccgtaagcgagagctaaagaccaatgaggaccatgtgtcccaggtgcagaaggtggagaagctcattgtggggaaaaagccgatcggatccctgcatcccgggctcggctgtgtgctctctctgccccaccgtcggttggtctgctactgccggctctttgaggttccagacccaaacaagccccagaaactcggactacaccagcgagaaatcttcctgttcaacgacctcctggtggtcaccaagatcttccagaagaagaagaactcggtgacgtacagcttccgacagtccttctccttgtacggcatgcaggtcctgctcttcgagaaccagtactaccccaatggcatccggctcacctcgtctgtccccggagcagatatcaaagtgttaataaacttcaacgcccccaaccctcaagaccggaagaaattcaccgatgacctgcgggagtccattgcggaagtccaagagatggagaagcacaggatagagtcggagctcgagaagcagaaaggcgtcgtgcggcccagcatgtcccagtgctctagcctcaaaaaggagtcgggcaacggaacactgagccgggcctgcctggacgacagctatgccagcggtgagggcctcaagcgcagcgccctcagcagctccctgcgggacctctcggaagccgggaagcgagggcgtcgcagcagtgcgggatcgctagagagcaatgtggaatttcagcctttcgagccactgcagccgtcagtgctgtgctcctaagccatgggacccgaggactgccccccggcgcctgcccaggcggaggccctttcagaaagggcgaagctcacgcctgactctgcgggccgcggggccgcgttcccagtggacagcgtggtgagccgtggccggacggcaggaggagaggggagccccctctggctgtgtgtcaccttggctggctggctggccagggttttgccgatttccctcctcacatccctcccaccctcggtcatatcattgagatcaccgtaccagcaaatctgcaaaccaagcactttgttaatctccagagagagcaaatacagcaatctagagtttagcctttttaaaaatgtgactttaacccgcccacctgtgtcctcccattggcgtggcatttctttggtgtctgctcagaaagaaacagcatgggaccgtgtgattgggtccaggctgggctgtcagggaagcgccctggtgtctccgactcatcaattctcgggttttttttaaaagccaggctccccaaagcactctttgtctcgcaattttgaggaggttgttccgacctggaagcgttaaactgctgcatgtcatctggcgtcagctgtgaggctgcgctctgcgtgttgtgcgtttgcaaaatgtattggcgtgactgtaaacacatcgttgaagtaaagcgattacatattgagtatgtcagcttttctcaccctcttctgtgctgggaggagagtctaactgtaacctttaggttgttccagaaaagattcaggccgtttggcacagaattgctctttctgaagggtagcagtaatgaataggaacagacagaaaaccccaggaggggctttcaggaaactctccctctccatcgccatcctgcaagctccacagcctcgggctgccagcctgtggcactgtcctcggtgtgtcctggagctgtgctgggcagcagggcctcgcgtaaggaactgctggaacccactgagcccttcccggggtttcccgccgcacttagtgtgttccttcaggctagtgaggtttcattctgtttatttgcaagcttatcaaaagtttgtggaagatacattgttgggttccagaggctggagtcatgctgcccacatcttgtttgggaaagcaaaagctcccagtgagctggtgtccccgtgtgtctttcatgggactgtcccctttagagatcccacctgtcagagccacagcttcggggtcctgtcatcacatcccctgggaggggaggcaggaggaggccgagggccccaggatgtctgcctggtcccttctcactgtgaggacgcccttggcacacctttctcaagtcacacaaattattgcagttacatacagaattgctgacaggagcaggagacgctgagggaaacagctggcaatgtgacaaaagtcttttctgggacaacaatcaaatcatgtatttgtatttttttaagtttaccaatgaattgtacaacaatgtaaaaataaagttcatcctaatatgctgtgc

SEQ ID NO: 10 - DNA, 1015 bp, Homo sapiens
  Homo sapiens peptidylprolyl isomerase C (cyclophilin C) PPIC (1)..(1015): representative cDNA sequence (NM_000943.2, as of 24 March 2003) for gene under Unigene ID Hs.110364
ggcacgaggcccgtcagctgtcccagagcctgtgtcgcgcccgtgccggtagcgcccgtgccggtagcgccgctgccaccgctcaccatgggcccgggtcctcggctgctgctacctctcgtgctttgcgtggggctcggcgcacttgtgttttcttcgggggccgagggcttccgcaagcgaggcccctcggtgacggccaaggtcttctttgatgtgaggattggagacaaagatgttggcagaattgtgattggcctctttggaaaagttgtgcccaagacagtggaaaattttgttgctctagcaacaggagagaaaggatatggatataaaggaagcaagtttcatcgtgtcatcaaggatttcatgattcaaggaggtgacatcaccactggagatggcactgggggtgtgagcatctatggtgagacatttccagatgagaacttcaagctgaagcactatggcattgggtgggtcagcatggccaacgctgggcctgacaccaatggctctcagttctttatcaccttgaccaagcccacctggttggacggcaaacatgtggtgtttggaaaagtcattgatgggatgacagtggtgcactccatagagctccaagcaactgatgggcatgaccgtccactcaccaactgctcgatcatcaacagtggcaagatagacgtgaaaacgccttttgtggttgagatcgctgattggtgacacaactggcagaaaacaaggatatgctttggcaggggtgtgtgtgtgtgtgtgtgtgtgtgtgtgttgtgttgtctttcaattatttgctttttttttttactttctttttgtattctatcccagatcacaggaaagttataaaaatcaaaccgtcaccctttagtttgcttgaactttagtaaaccacctgcttagggactttgaacttaaatatatccccttcctcaagtggtgctattttaaaactaaaaaaaactttgaattggcaaaaaaaaaaaaaaaaaaa

SEQ ID NO: 11 - DNA, 2565 bp, Homo sapiens
  Homo sapiens LAG1 longevity assurance homolog 1 (1)..(2565): representative cDNA sequence (NM_021267.1, as of 24 March 2003) for gene under Unigene ID Hs.348258
acgcggggcgcgcggctccgtcggctaccgcgggcgggcgcaggcgacgggcacggcgggcgagcgggcggtatggcggcggcggggcccgcggcggggccgacggggcccgagcccatgccgagctacgcgcagctagtgcagcgcggctggggcagcgcgctggcggcggcgcggggctgcacggactgcggctgggggctggcgcgtcgcggcctggctgagcacgcgcacctggcgccgcccgagctgctgctgctggcgctcggcgcgctgggctggaccgcgctgcgctccgcggccactgcgcgcctctttcggcccctggcgaagcggtgctgcctccagcccagagatgccgccaagatgcccgagagcgcttggaagtttctcttctacctgggcagctggagctacagtgcctacctgctgtttggcaccgactaccccttcttccatgacccaccatctgtcttctacgactggacgccgggcatggcagtgccacgggacattgcagccgcctacctgctccagggaagcttctatggccactccatctacgctacgctatacatggacacctggcgcaaggactcggtggtcatgctgctccaccacgtggtcactctcatcctcatcgtctcctcctacgccttccggtaccacaatgtgggcatccttgtgctcttcctgcacgatatcagtgacgtgcagcttgagttcaccaagctcaacatttacttcaagtcccgcggcggctcctaccatcggctgcatgccttggcagcagacttgggctgcctcagcttcggcttcagctggttctggttccgcctctactggttcccgctcaaggtcctgtatgccaccagtcactgcagtctgcgcacggtgcctgacatccccttctacttcttcttcaatgcgctcctgctgctgctcacccttatgaacctctactggttcctgtacatcgtggcgtttgcagccaaggtgttgacaggccaggtgcacgagctgaaggacctgcgggagtatgacacagccgaggcccagagcctgaagcccagcaaagccgagaagccactgaggaacggcctggtgaaggacaagcgcttctgaacccctcggccccgcccccgtggacccggccccaccccgaataccccggccacgctccccgtccttggccgcccctccaccccctccaactctgctcctctagggccgccgccacctcccctgggaccccgccccctcatcctgcctccatttcccggccacgccccccaggacccctgcccctccggggacaccggccccgccctcagcccactggtcccgggccgccgcggaccctgcgcactctctggtcatcgcctgggaggaagatgccaccgccgcagcaaggtccctgcggccaccacctcctcctcctcctggccctgctgctgccctcgctgcccctgacccgcgcccccgtgcccccaggcccagccgccgccctgctccaggctctaggactgcgcgatgagccccagggtgcccccaggctccggccggttcccccggtcatgtggcgcctgtttcgacgccgggacccccaggagaccaggtctggctcgcggcggacgtccccaggggtcaccctgcaaccgtgccacgtggaggagctgggggtcgccggaaacatcgtgcgccacatcccggaccgcggtgcgcccacccgggcctcggagcctgtctcggccgcggggcattgccctgagtggacagtcgtcttcgacctgtcggctgtggaacccgctgagcgcccgagccgggcccgcctggagctgcgtttcgcggcggcggcggcggcagccccggagggcggctgggagctgagcgtggcgcaagcgggccagggcgcgggcgcggaccccgggccggtgctgctccgccagttggtgcccgccctggggccgccagtgcgcgcggagctgctgggcgccgcttgggctcgcaacgcctcatggccgcgcagcctccgcctggcgctggcgctacgcccccgggcccctgccgcctgcgcgcgcctggccgaggcctcgctgctgctggtgaccctcgacccgcgcctgtgccaccccctggcccggccgcggcgcgacgccgaacccgtgttgggcggcggccccgggggcgcttgtcgcgcgcggcggctgtacgtgagcttccgcgaggtgggctggcaccgctgggtcatcgcgccgcgcggcttcctggccaactactgccagggtcagtgcgcgctgcccgtcgcgctgtcggggtccggggggccgccggcgctcaaccacgctgtgctgcgcgcgctcatgcacgcggccgccccgggagccgccgacctgccctgctgcgtgcccgcgcgcctgtcgcccatctccgtgctcttctttgacaacagcgacaacgtggtgctgcggcagtatgaggacatggtggtggacgagtgcggctgccgctaacccggggcgggcagggacgcgggcccaacaataaatgccgcgtggtctgctc

SEQ ID NO: 12 - DNA, 2880 bp, Homo sapiens
  Homo sapiens nuclear factor of activated T-cells, cytoplasmic, calcineurin-dependent 4 (NFATC4) (1)..(2880): representative cDNA sequence (NM_004554.1, as of 24 March 2003) for gene under Unigene ID Hs.77810
gcttctggagggaggcggcagcgacggaggagggggcttctcagagaaagggagggagggagccacccgggtgaagatacagcagcctcctgaactcccccctcccacccaggccgggacctgggggctcctgccggatccatgggggcggccagctgcgaggatgaggagctggaatttaagctggtgttcggggaggaaaaggaggcccccccgctgggcgcggggggattgggggaagaactggactcagaggatgccccgccatgctgccgtctggccttgggagagccccctccctatggcgctgcacctatcggtattccccgacctccaccccctcggcctggcatgcattcgccaccgccgcgaccagccccctcacctggcacctgggagagccagcccgccaggtcggtgaggctgggaggaccaggagggggtgctgggggtgctgggggtggccgtgttctcgagtgtcccagcatccgcatcacctccatctctcccacgccggagccgccagcagcgctggaggacaaccctgatgcctggggggacggctctcctagagattaccccccaccagaaggctttgggggctacagagaagcaggggcccagggtgggggggccttcttcagcccaagccctggcagcagcagcctgtcctcgtggagcttcttctccgatgcctctgacgaggcagccctgtatgcagcctgcgacgaggtggagtctgagctaaatgaggcggcctcccgctttggcctgggctccccgctgccctcgccccgggcctcccctcggccatggacccccgaagatccctggagcctgtatggtccaagccccggaggccgagggccagaggatagctggctactcctcagtgctcctgggcccaccccagcctccccgcggcctgcctctccatgtggcaagcggcgctattccagctcgggaaccccatcttcagcctccccagctctgtcccgccgtggcagcctgggggaagaggggtctgagccacctccaccacccccattgcctctggcccgggacccgggctcccctggtccctttgactatgtgggggccccaccagctgagagcatccctcagaagacacggcggacttccagcgagcaggcagtggctctgcctcggtctgaggagcctgcctcatgcaatgggaagctgcccttgggagcagaggagtctgtggctcctccaggaggttcccggaaggaggtggctggcatggactacctggcagtgccctccccactcgcttggtccaaggcccggattgggggacacagccctatcttcaggacctctgccctacccccactggactggcctctgcccagccaatatgagcagctggagctgaggatcgaggtacagcctagagcccaccaccgggcccactatgagacagaaggcagccgtggagctgtcaaagctgcccctggcggtcaccccgtagtcaagctcctaggctacagtgagaagccactgaccctacagatgttcatcggcactgcagatgaaaggaacctgcggcctcatgccttctatcaggtgcaccgtatcacaggcaagatggtggccacggccagctatgaagccgtagtcagtggcaccaaggtgttggagatgactctgctgcctgagaacaacatggcggccaacattgactgcgcgggaatcctgaagcttcggaattcagacattgagcttcggaagggtgagacggacatcgggcgcaaaaacacacgtgtacggctggtgttccgggtacacgtgccccagggcggcgggaaggtcgtctcagtacaggcagcatcggtgcccatcgagtgctcccagcgctcagcccaggagctgccccaggtggaggcctacagccccagtgcctgctctgtgagaggaggcgaggaactggtactgaccggctccaacttcctgccagactccaaggtggtgttcattgagaggggtcctgatgggaagctgcaatgggaggaggaggccacagtgaaccgactgcagagcaacgaggtgacgctgaccctgactgtccccgagtacagcaacaagagggtttcccggccagtccaggtctacttttatgtctccaatgggcggaggaaacgcagtcctacccagagtttcaggtttctgcctgtgatctgcaaagaggagcccctaccggactcatctctgcggggtttcccttcagcatcggcaaccccctttggcactgacatggacttctcaccacccaggcccccctacccctcctatccccatgaagaccctgcttgcgaaactccttacctatcagaaggcttcggctatggcatgccccctctgtacccccagacggggcccccaccatcctacagaccgggcctgcggatgttccctgagactaggggtaccacaggttgtgcccaaccacctgcagtttccttccttccccgccccttccctagtgacccgtatggagggcggggctcctctttccccctggggctgccattctctccgccagccccctttcggccgcctcctcttcctgcatccccaccgcttgaaggccccttcccttcccagagtgatgtgcatcccctacctgctgagggatacaataaggtagggccaggctatggccctggggagggggctccggagcaggagaaatccaggggtggctacagcagcggctttcgagacagtgtccctatccagggtatcacgctggaggaagtgagtgagatcattggccgagacctgagtggcttccctgcacctcctggagaagagcctcctgcctgaaccacgtgaactgtcatcacctggcaaccc

SEQ ID NO: 13 - DNA, 2546 bp, Homo sapiens
  H.sapiens mRNA for nuclear receptor (1)..(2546): representative cDNA sequence (X89894.1, as of 24 March 2003)  for gene under Unigene ID Hs.80561
gagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctccagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgctcttgtccgagctttaacagactcaacacccagagatcttgattattccagagtaagttttatgatttcctgctttcaaatgaatgatcagggtctctatttatggctactagtaataagagttgattgaatgattttgtgtctggcaccatgttagacagttttcatactttttctatatttctcgcttcatttagcaattcagtgcatccattgcagcaaataatttttgccttattgaatctctaaatgccttaacaagtgaccctgacagtgctgcacctgtcatacacattgttgcaggattcctggtggttgtgccaatgaaaatctgcacagacaaactacaatttgtagatttatctcgtgatctagacaaagtgactactgttttttttcatattgtgttcaaaccatctgggtgagcctcaagttattactaagcagtttatccaattgcatcagcattgattgacctgctgcttattcagagggattagaccattggtggagagaagcgagctctggctgtgacatcagttgtcaggagaactgggatggaacgccacctctgctacttcctgcctgtgtcacctggttcctgcttttggctctctcctgcctacagtggaaatgactacaatatttaacctcaga

SEQ ID NO: 14 - DNA, 1677 bp, Homo sapiens
  Homo sapiens proline dehydrogenase (oxidase) 2 (PRODH2) (1)..(1677): representative cDNA sequence (NM_021232.1, as of 24 March 2003) for gene under Unigene ID Hs.128834
tagagatggggtctcactatgtcgcccagggtagtctcaaactcctcggtcttggcctcccaaagtgttgggattacaaacgtgagaaccgtattttcaaatgtattcaataacactacagcgtttccaattttaagaggaagcaattgccacaaaatcactgcccctggtctgggcaaggggcagttggtgaacttgctgcctccagagaaccttccctggtgtggaggcagccagggacccaggatgctccggacctgttacgtgctctgttcccaagctggtcccccctccaggggctggcagtccctgagctttgatggcggggccttccaccttaagggcacaggagagctgacacgggccttgctggttctccggctgtgtgcctggcccccactcgtcactcacgggctgttgctccaggcctggtctcggcgactcctgggctcccggctctcaggcgcatttctccgagcatccgtctatgggcagtttgtggctggtgagacagcagaggaggtgaagggctgcgtgcagcagctgcggaccctcagcctccgaccactgctggcagtgcccactgaggaggagccggactctgctgccaagagtggtgaggcgtggtatgaggggaacctcggtgctatgctgcggtgtgtggacctgtcacggggcctcctggagccccccagcctggctgaggccagcctcatgcagctgaaggtgacggcgctgaccagtactcggctctgtaaggagctagcctcgtgggtcagaaggccaggagcctccttggagctgagccccgagaggctggctgaagctatggactctgggcagaacctccaggtctcctgcctcaatgctgagcagaaccagcacctccgggcctccctcagccgcctgcatcgggtggcacagtatgcccgggcccagcacgtgcggctcctggtggatgcggagtacacctcactgaaccctgcgctctcgctgctggtggctgccctggctgtgcgctggaacagcccgggtgaaggcgggccctgggtgtggaacacctaccaggcctgtctaaaggacacattcgagcggctggggagggatgcagaggctgcgcacagggccggcctggccttcggagtgaagctggtacgaggtgcatatctggacaaggagagagcggtggcccagctccatgggatggaagaccccactcagcctgactatgaggccaccagtcagagttacagccgctgcctggaactgatgctgacgcacgtggcccgccatggccccatgtgccacctcatggtggcttcccacaatgaggaatctgttcgccaggcaaccaagcgcatgtgggagctgggcattcctctggatgggactgtctgtttcggacaacttctgggcatgtgtgaccacgtctctctagcactggggcaggccggctatgtagtgtataagtccattccctatggctccttggaggaggtaatcccctacctgatccggagggcccaggagaaccggagcgtgcttcagggtgcccgcagggaacaggagctgctcagccaagaactgtggcggcggctgctgccaggatgccgaaggataccccactagcacccctgagggggtcatgtggtcaataaaagtccttaggtgctgcct

SEQ ID NO: 15 - DNA, 2261 bp, Homo sapiens
  Homo sapiens protein kinase C, iota (PRKCI) (1)..(2261): representative cDNA sequence (NM_002740.1, as of 24 March 2003) for gene under Unigene ID Hs.1904
ccgcggttccggctgctccggcgaggcgacccttgggtcggcgctgcgggcgaggtgggcaggtaggtgggcggacggccgcggttctccggcaagcgcaggcggcggagtcccccacggcgcccgaagcgccccccgcacccccggcctccagcgttgaggcgggggagtgaggagatgccgacccagagggacagcagcaccatgtcccacacggtcgcaggcggcggcagcggggaccattcccaccaggtccgggtgaaagcctactaccgcggggatatcatgataacacattttgaaccttccatctcctttgagggcctttgcaatgaggttcgagacatgtgttcttttgacaacgaacagctcttcaccatgaaatggatagatgaggaaggagacccgtgtacagtatcatctcagttggagttagaagaagcctttagactttatgagctaaacaaggattctgaactcttgattcatgtgttcccttgtgtaccagaacgtcctgggatgccttgtccaggagaagataaatccatctaccgtagaggtgcacgccgctggagaaagctttattgtgccaatggccacactttccaagccaagcgtttcaacaggcgtgctcactgtgccatctgcacagaccgaatatggggacttggacgccaaggatataagtgcatcaactgcaaactcttggttcataagaagtgccataaactcgtcacaattgaatgtgggcggcattctttgccacaggaaccagtgatgcccatggatcagtcatccatgcattctgaccatgcacagacagtaattccatataatccttcaagtcatgagagtttggatcaagttggtgaagaaaaagaggcaatgaacaccagggaaagtggcaaagcttcatccagtctaggtcttcaggattttgatttgctccgggtaataggaagaggaagttatgccaaagtactgttggttcgattaaaaaaaacagatcgtatttatgcaatgaaagttgtgaaaaaagagcttgttaatgatgatgaggatattgattgggtacagacagagaagcatgtgtttgagcaggcatccaatcatcctttccttgttgggctgcattcttgctttcagacagaaagcagattgttctttgttatagagtatgtaaatggaggagacctaatgtttcatatgcagcgacaaagaaaacttcctgaagaacatgccagattttactctgcagaaatcagtctagcattaaattatcttcatgagcgagggataatttatagagatttgaaactggacaatgtattactggactctgaaggccacattaaactcactgactacggcatgtgtaaggaaggattacggccaggagatacaaccagcactttctgtggtactcctaattacattgctcctgaaattttaagaggagaagattatggtttcagtgttgactggtgggctcttggagtgctcatgtttgagatgatggcaggaaggtctccatttgatattgttgggagctccgataaccctgaccagaacacagaggattatctcttccaagttattttggaaaaacaaattcgcataccacgttctctgtctgtaaaagctgcaagtgttctgaagagttttcttaataaggaccctaaggaacgattgggttgtcatcctcaaacaggatttgctgatattcagggacacccgttcttccgaaatgttgattgggatatgatggagcaaaaacaggtggtacctccctttaaaccaaatatttctggggaatttggtttggacaactttgattctcagtttactaatgaacctgtccagctcactccagatgacgatgacattgtgaggaagattgatcagtctgaatttgaaggttttgagtatatcaatcctcttttgatgtctgcagaagaatgtgtctgatcctcatttttcaaccatgtattctactcatgttgccatttaatgcatggataaacttgctgcaagcctggatacaattaaccattttatatttgccacctacaaaaaaacacccaatatcttctcttgtagactatatgaatcaattattacatctgttttactatgaaaaaaaaattaatactactagcttccagacaatcatgtcaaaatttagttgaactggtttttcagtttttaaaaggcctacagatgagtaatgaagttaccttttttgtttaaaaaaaaaaaag

SEQ ID NO: 16 - DNA, 6880 bp, Homo sapiens
  Homo sapiens mRNA for ATP-binding cassette transporter-1 (ABC-1) (1)..(6880): representative cDNA sequence (AJ012376.1, as of 24 March 2003)  for gene under Unigene ID Hs.211562
caaacatgtcagctgttactggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggccgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccaccaaacaaatgaagaaacacctaaagctggccaaggacagttctgcagatcgatttctcaacagcttgggaagatttatgacaggactggacaccagaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctccgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacagaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttccaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagagggactagactttcctttgcaccatgtgaagtgttgtggagaaaagagccagaagttgatgtgggaagaagtaaactggatactgtactgatactattcaatgcaatgcaattcaatg

SEQ ID NO: 17 - DNA, 2930 bp, Homo sapiens
  Homo sapiens ATP-binding cassette, sub-family G (WHITE), member 1(ABCG1), transcript variant 1 (1)..(2930): representative cDNA sequence (NM_004915, as of 24 March 2003)  for gene under Unigene ID Hs.10237
gaattccggtttcttcctaaaaaatgtctgatggccgctttctcggtcggcaccgccatgaatgccagcagttactctgcagagatgacggagcccaagtcggtgtgtgtctcggtggatgaggtggtgtccagcaacatggaggccactgagacggacctgctgaatggacatctgaaaaaagtagataataacctcacggaagcccagcgcttctcctccttgcctcggagggcagctgtgaacattgaattcagggacctttcctattcggttcctgaaggaccctggtggaggaagaaaggatacaagaccctcctgaaaggaatttccgggaagttcaatagtggtgagttggtggccattatgggtccttccggggccgggaagtccacgctgatgaacatcctggctggatacagggagacgggcatgaagggggccgtcctcatcaacggcctgccccgggacctgcgctgcttccggaaggtgtcctgctacatcatgcaggatgacatgctgctgccgcatctcactgtgcaggaggccatgatggtgtcggcacatctgaagcttcaggagaaggatgaaggcagaagggaaatggtcaaggagatactgacagcgctgggcttgctgtcttgcgccaacacgcggaccgggagcctgtcaggtggtcagcgcaagcgcctggccatcgcgctggagctggtgaacaaccctccagtcatgttcttcgatgagcccaccagcggcctggacagcgcctcctgcttccaggtggtctcgctgatgaaagggctcgctcaagggggtcgctccatcatttgcaccatccaccagcccagcgccaaactcttcgagctgttcgaccagctttacgtcctgagtcaaggacaatgtgtgtaccggggaaaagtctgcaatcttgtgccatatttgagggatttgggtctgaactgcccaacctaccacaacccagcagattttgtcatggaggttgcatccggcgagtacggtgatcagaacagtcggctggtgagagcggttcgggagggcatgtgtgactcagaccacaagagagacctcgggggtgatgccgaggtgaacccttttctttggcaccgcccctctgaagaggtaaagcagacaaaacgattaaaggggttgagaaaggactcctcgtccatggaaggctgccacagcttctctgccagctgcctcacgcagttctgcatcctcttcaagaggaccttcctcagcatcatgagggactcggtcctgacacacctgcgcatcacctcgcacattgggatcggcctcctcattggcctgctgtacttggggatcgggaacgaaaccaagaaggtcttgagcaactccggcttcctcttcttctccatgctgttcctcatgttcgcggccctcatgcctactgttctgacatttcccctggagatgggagtctttcttcgggaacacctgaactactggtacagcctgaaggcctactacctggccaagaccatggcagacgtgccctttcagatcatgttcccagtggcctactgcagcatcgtgtactggatgacgtcgcagccgtccgacgccgtgcgctttgtgctgtttgccgcgctgggcaccatgacctccctggtggcacagtccctgggcctgctgatcggagccgcctccacgtccctgcaggtggccactttcgtgggcccagtgacagccatcccggtgctcctgttctcggggttcttcgtcagcttcgacaccatccccacgtacctacagtggatgtcctacatctcctatgtcaggtatgggttcgaaggggtcatcctctccatctatggcttagaccgggaagatctgcactgtgacatcgacgagacgtgccacttccagaagtcggaggccatcctgcgggagctggacgtggaaaatgccaagctgtacctggacttcatcgtactcgggattttcttcatctccctccgcctcattgcctatttggtcctcaggtacaaaatccgggcagagaggtaaaacacctgaatgccaggaaacaggaagattagacactgtggccgagggcacgtctagaatcgaggaggcaagcctgtgcccgaccgacgacacagagactcttctgatccaacccctagaaccgcgttgggtttgtgggtgtctcgtgctcagccactctgcccagctgggttggatcttctctccattcccctttctagctttaactaggaagatgtaggcagattggtggttttttttttttttttaacatacagaattttaaataccacaactggggcagaatttaaagctgcaacacagctggtgatgagaggcttcctcagtccagtcgctccttagcaccaggcaccgtgggtcctggatggggaactgcaagcagcctctcagctgatggctgcacagtcagatgtctggtggcagagagtccgagcatggagcgattccattttatgactgttgtttttcacattttcatctttctaaggtgtgtctcttttccaatgagaagtcatttttgcaagccaaaagtcgatcaatcgcattcattttaagaaattatacctttttagtacttgctgaagaatgattcagggtaaatcacatactttgtttagagaggcgaggggtttaacccgagtcacccagctggtctcatacatagacagcacttgtgaaggattgaatgcaggttccaggtggagggaagacgtggacaccatctccactgagccatgcagacatttttaaaagctatacacaaaattgtgagaagacattggccaactctttcaaagtctttctttttccacgtgcttcttattttaagcgaaatatattgtttgtttcttcctaaaaaaaaaaaaaaaaaaaaa

SEQ ID NO: 18 - DNA, 417 bp, Homo sapiens
  Homo sapiens apolipoprotein C-I (APOC1) (1)..(417): representative cDNA sequence (NM_001645.2, as of 24 March 2003) for gene under Unigene ID Hs.268571
acctcccaaccaagccctccagcaaggattcaggagtgcccctcgggcctcgccatgaggctcttcctgtcgctcccggtcctggtggtggttctgtcgatcgtcttggaaggcccagccccagcccaggggaccccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatgaggacctgaagggtgacatccaggaggggcctctgaaatttcccacaccccagcgcctgtgctgaggactcccgccatgtggccccaggtgccaccaataaaaatcctaccg

SEQ ID NO: 19 - DNA, 1156 bp, Homo sapiens
  Homo sapiens apolipoprotein E (APOE) (1)..(1156): representative cDNA sequence (NM_000041.1, as of 24 March 2003) for gene under Unigene ID Hs.169401
cgcagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgc

SEQ ID NO: 20 - DNA, 1768 bp, Homo sapiens
  Homo sapiens acid sphingomyelinase-like phosphodiesterase (ASM3A), (1)..(1768): representative cDNA sequence (NM_006714.1 as of 24 March 2003)  for gene under Unigene ID Hs.42945
ggcacgaggctcaggcctgacggtccgagtggagctgcgggacagcccgaacctccaggtcagccccgcggccctccatggcgctggtgcgcgcactcgtctgctgcctgctgactgcctggcactgccgctccggcctcgggctgcccgtggcgcccgcaggcggcaggaatcctcctccggcgataggacagttttggcatgtgactgacttacacttagaccctacttaccacatcacagatgaccacacaaaagtgtgtgcttcatctaaaggtgcaaatgcctccaaccctggcccttttggagatgttctgtgtgattctccatatcaacttattttgtcagcatttgattttattaaaaattctggacaagaagcatctttcatgatatggacaggggatagcccacctcatgttcctgtacctgaactctcaacagacactgttataaatgtgatcactaatatgacaaccaccatccagagtctctttccaaatctccaggttttccctgcgctgggtaatcatgactattggccacaggatcaactgcctgtagtcaccagtaaagtgtacaatgcagtagcaaacctctggaaaccatggctagatgaagaagctattagtactttaaggaaaggtggtttttattcacagaaagttacaactaatccaaaccttaggatcatcagtctaaacacaaacttgtactacggcccaaatataatgacactgaacaagactgacccagccaaccagtttgaatggctagaaagtacattgaacaactctcagcagaataaggagaaggtgtatatcatagcacatgttccagtggggtatctgccatcttcacagaacatcacagcaatgagagaatactataatgagaaattgatagatatttttcaaaaatacagtgatgtcattgcaggacaattttatggacacactcacagagacagcattatggttctttcagataaaaaaggaagtccagtaaattctttgtttgtggctcctgctgttacaccagtgaagagtgttttagaaaaacagaccaacaatcctggtatcagactgtttcagtatgatcctcgtgattataaattattggatatgttgcagtattacttgaatctgacagaggcgaatctaaagggagagtccatctggaagctggagtatatcctgacccagacctacgacattgaagatttgcagccggaaagtttatatggattagctaaacaatttacaatcctagacagtaagcagtttataaaatactacaattacttctttgtgagttatgacagcagtgtaacatgtgataagacatgtaaggcctttcagatttgtgcaattatgaatcttgataatatttcctatgcagattgcctcaaacagctttatataaagcacaattactagtatttcacagtttttgctaatagaaaatgctgattctgattctgagatcaatttgtgggaattttacataaatctttgttaattactgagtgggcaagtagacttcctgtctttgctttcttttttttttctttttgatgccttaatgtagatatctttatcattctgaattgtattatatatttaaagtgctcattaatagaatgatggatgtaaattggatgtaaatattcagtttatataattatatctaatttgtacccttgttgaaattgtcatttatacaataaagcgaattctttatctctaaaaaaaaaaaaaaaaaaaaa

SEQ ID NO: 21 - DNA, 1170 bp, Homo sapiens
  Homo sapiens Cdc42 effector protein 4 mRNA (1)..(1170): representative cDNA sequence (AF099664.1) as of 24 March 2003)  for gene under Unigene ID Hs.3903
actggacttggacttcagatctgaccccagacctgccggctacctcgggagggcccacctccccgcccatccagcaagatgccaatcctcaagcaactggtgtccagctcggtgcactccaagcgccgttcccgagcggacctcacggccgagatgatcagcgccccgctggggactttccgccacaccatgcacgttggccgggccggagacgcctttggggacacctccttcctcaatagcaaggctggcgagcccgacggcgagtccttggacgaacagccctcttcttcatcttccaaacgcagtctcctgtccaggaagttccggggcagcaagcggtcacagtcggtgaccaggggggagcgggagcagcgtgacatgctgggctccctgcgggactcggccctgtttgtcaagaatgccatgtccctcccccagctcaatgagaaggaggccgcggagaagggcaccagtaagctgcccaagagcctgtcatccagccccgtgaagaaggccaatgacggggagggcggcgatgaggaggcgggcacggaggaggcagtgccccgtcggaatggggccgcgggtccacattcccctgaccccctcctcgatgagcaggcctttggggatctgacagatctgcctgtcgtgcccaaggccacgtacgggctgaagcatgcggagtccatcatgtccttccacatcgacctggggccctccatgctgggtgacgtcctcagcatcatggacaaggaggagtgggaccccgaggagggggagggtggttaccatggcgatgagggcgccgctggcaccatcacccaggctcccccgtacgccgtggcggcccctcccctggcaaggcaggaaggcaaggctggcccagacttgccctccctcccctcccatgctctggaggatgaggggtgggcaacagcggcccccagccccggctcaacccgcagcatgggcagccacaccacacgggacagcagctccctctccagctgcacctcaggcatcctggaggagcgcagccctgccttccgggggccggacagggcccgggctgctgtctcaagacagccaccagacaaggagttctccttcatggatgaggaggaggaggatgaaatcgtgtgaggcggacagtgggtggccacc

SEQ ID NO: 22 - DNA, 1679 bp, Homo sapiens
  Homo sapiens chemokine (C-X-C motif) receptor 4 (CXCR4) (1)..(1679): representative cDNA sequence (NM_003467) as of 24 March 2003)  for gene under Unigene ID Hs.89414
gtttgttggctgcggcagcaggtagcaaagtgacgccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttacttagtgttatg

SEQ ID NO: 23 - DNA, 8461 bp, Homo sapiens
  Homo sapiens fatty acid synthase (FASN) (1)..(8461): representative cDNA sequence (NM_004104.3) as of 24 March 2003) for gene under Unigene ID Hs.213289
gagggagccagagagacggcagcggccccggcctccctctccgccgcgcttcagcctcccgctccgccgcgctccagcctcgctctccgccgcccgcaccgccgcccgcgccctcaccagagcagccatggaggaggtggtgattgccggcatgtccgggaagctgccagagtcggagaacttgcaggagttctgggacaacctcatcggcggtgtggacatggtcacggacgatgaccgtcgctggaaggcggggctctacggcctgccccggcggtccggcaagctgaaggacctgtctaggtttgatgcctccttcttcggagtccaccccaagcaggcacacacgatggaccctcagctgcggctgctgctggaagtcacctatgaagccatcgtggacggaggcatcaacccagattcactccgaggaacacacactggcgtctgggtgggcgtgagcggctctgagacctcggaggccctgagccgagaccccgagacactcgtgggctacagcatggtgggctgccagcgagcgatgatggccaaccggctctccttcttcttcgacttcagagggcccagcatcgcactggacacagcctgctcctccagcctgatggccctgcagaacgcctaccaggccatccacagcgggcagtgccctgccgccatcgtggggggcatcaatgtcctgctgaagcccaacacctccgtgcagttcttgaggctggggatgctcagccccgagggcacctgcaaggccttcgacacagcggggaatgggtactgccgctcggagggtgtggtggccgtcctgctgaccaagaagtccctggcccggcgggtgtacgccaccatcctgaacgccggcaccaatacagatggcttcaaggagcaaggcgtgaccttcccctcaggggatatccaggagcagctcatccgctcgttgtaccagtcggccggagtggcccctgagtcatttgaatacatcgaagcccacggcacaggcaccaaggtgggcgacccccaggagctgaatggcatcacccgagccctgtgcgccacccgccaggagccgctgctcatcggctccaccaagtccaacatggggcacccggagccagcctcggggctggcagccctggccaaggtgctgctgtccctggagcacgggctctgggcccccaacctgcacttccatagccccaaccctgagatcccagcgctgttggatgggcggctgcaggtggtggaccagcccctgcccgtccgtggcggcaacgtgggcatcaactcctttggcttcgggggctccaacgtgcacatcatcctgaggcccaacacgcagccgccccccgcacccgccccacatgccaccctgccccgtctgctgcgggccagcggacgcacccctgaggccgtgcagaagctgctggagcagggcctccggcacagccaggacctggctttcctgagcatgctgaacgacatcgcggctgtccccgccaccgccatgcccttccgtggctacgctgtgctgggtggtgagcgcggtggcccagaggtgcagcaggtgcccgctggcgagcgcccgctctggttcatctgctctgggatgggcacacagtggcgcgggatggggctgagcctcatgcgcctggaccgcttccgagattccatcctacgctccgatgaggctgtgaagccattcggcctgaaggtgtcacagctgctgctgagcacagacgagagcacctttgatgacatcgtccattcgtttgtgagcctgactgccatccagataggcctcatagacctgctgagctgcatggggctgaggccagatggcatcgtcggccactccctgggggaggtggcctgtggctacgccgacggctgcctgtcccaggaggaggccgtcctcgctgcctactggaggggacagtgcatcaaagaagcccatctcccgccgggcgccatggcagccgtgggcttgtcctgggaggagtgtaaacagcgctgccccccggcggtggtgcccgcctgccacaactccaaggacacagtcaccatctcgggacctcaggccccggtgtttgagttcgtggagcagctgaggaaggagggtgtgtttgccaaggaggtgcggaccggcggtatggccttccactcctacttcatggaggccatcgcacccccactgctgcaggagctcaagaaggtgatccgggagccgaagccacgttcagcccgctggctcagcacctctatccccgaggcccagtggcacagcagcctggcacgcacgtcctccgccgagtacaatgtcaacaacctggtgagccctgtgctgttccaggaggccctgtggcacgtgcctgagcacgcggtggtgctggagatcgcgccccacgccctgctgcaggctgtcctgaagcgtggcctgaagccgagctgcaccatcatccccctgatgaagaaggatcacagggacaacctggagttcttcctggccggcatccggaggctgcacctctcaggcatcgacgccaaccccaatgccttgttcccacctgtggagttcccagctccccgaggaactcccctcatctccccactcatcaagtgggaccacagcctggcctgggacgtgccggccgccgaggacttccccaacggttcaggttccccctcagccgccatctacaacatcgacaccagctccgagtctcctgaccactacctggtggaccacaccctcgacggtcgcgtcctcttccccgccactggctacctgagcatagtgtggaagacgctggcccgacccctgggcctgggcgtcgagcagctgcctgtggtgtttgaggatgtggtgctgcaccaggccaccatcctgcccaagactgggacagtgtccctggaggtacggctcctggaggcctcccgtgccttcgaggtgtcagagaacggcaacctggtagtgagtgggaaggtgtaccagtgggatgaccctgaccccaggctcttcgaccacccggaaagccccacccccaaccccacggagcccctcttcctggcccaggctgaagtttacaaggagctgcgtctgcgtggctacgactacggccctcatttccagggcatcctggaggccagcctggaaggtgactcggggaggctgctgtggaaggataactgggtgagcttcatggacaccatgctgcagatgtccatcctgggctcggccaagcacggcctgtacctgcccacccgtgtcaccgccatccacatcgaccctgccacccacaggcagaagctgtacacactgcaggacaaggcccaagtggctgacgtggtggtgagcaggtggctgagggtcacagtggccggaggcgtccacatctccgggctccacactgagtcggccccgcggcggcagcaggagcagcaggtgcccatcctggagaagttttgcttcactccccacacggaggaggggtgcctgtctgagcgcgctgccctgcaggaggagctgcaactgtgcaaggggctggtgcaggcactgcagaccaaggtgacccagcaggggctgaagatggtggtgcccggactggatggggcccagatcccccgggacccctcacagcaggaactgccccggctgttgtcggctgcctgcaggcttcagctcaacgggaacctgcagctggagctggcgcaggtgctggcccaggagaggcccaagctgccagaggaccctctgctcagcggcctcctggactccccggcactcaaggcctgcctggacactgccgtggagaacatgcccagcctgaagatgaaggtggtggaggtgctggccggccacggtcacctgtattcccgcatcccaggcctgctcagcccccatcccctgctgcagctgagctacacggccaccgaccgccacccccaggccctggaggctgcccaggccgagctgcagcagcacgacgttgcccagggccagtgggatcccgcagaccctgcccccagcgccctgggcagcgccgacctcctggtgtgcaactgtgctgtggctgccctcggggacccggcctcagctctcagcaacatggtggctgccctgagagaagggggctttctgctcctgcacacactgctccgggggcacccctcgggacatgtggccttcctcacctccactgagccgcagtatggccagggcatcctgagccaggacgcgtgggagagcctcttctccagggtgtccgtgcgcctggtgggcctgaagaagtccttctacggctccacgctcttcctgtgccgccggcccaccccgcaggacagccccatcttcctgccggtggacgataccagcttccgctgggtggagtctctgaagggcatcctggctgacgaagactcttcccggcctgtgtggctgaaggccatcaactgtgccacctcgggcgtggtgggcttggtgaactgtctccgccgagagcccggcggaacgctccggtgtgtgctgctctccaacctcagcagcacctcccacgtcccggaggtggacccgggctccgcagaactgcagaaggtgttgcagggagacctggtgatgaacgtctaccgcgacggggcctggggggctttccgccacttcctgctggaggaggacaagcctgaggagccgacggcacatgcctttgtgagcaccctcacccggggggacctgtcctccatccgctgggtctgctcctcgctgcgccatgcccagcccacctgccctggcgcccagctctgcacggtctactacgcctccctcaacttccgcgacatcatgctggccactggcaagctgtcccctgatgccatcccagggaagtggacctcccaggacagcctgctaggtatggagttctcgggccgagacgccagcggcaagcgtgtgatgggactggtgcctgccaagggcctggccacctctgtcctgctgtcaccggacttcctctgggatgtgccttccaactggacgctggaggaggcggcctcggtgcctgtcgtctacagcacggcctactacgcgctggtggtgcgtgggcgggtgcgccccggggagacgctgctcatccactcgggctcgggcggcgtgggccaggccgccatcgccatcgccctcagtctgggctgccgcgtcttcaccaccgtggggtcggctgagaagcgggcgtacctccaggccaggttcccccagctcgacagcaccagcttcgccaactcccgggacacatccttcgagcagcatgtgctgtggcacacgggcgggaagggcgttgacctggtcttgaactccttggcggaagagaagctgcaggccagcgtgaggtgcttggctacgcacggtcgcttcctggaaattggcaaattcgacctttctcagaaccacccgctcggcatggctatcttcctgaagaacgtgacattccacggggtcctactggatgcgttcttcaacgagagcagtgctgactggcgggaggtgtgggcgcttgtgcaggccggcatccgggatggggtggtacggcccctcaagtgcacggtgttccatggggcccaggtggaggacgccttccgctacatggcccaagggaagcacattggcaaagtcgtcgtgcaggtgcttgcggaggagccggaggcagtgctgaagggggccaaacccaagctgatgtcggccatctccaagaccttctgcccggcccacaagagctacatcatcgctggtggtctgggtggcttcggcctggagttggcgcagtggctgatacagcgtggggtgcagaagctcgtgttgacttctcgctccgggatccggacaggctaccaggccaagcaggtccgccggtggaggcgccagggcgtacaggtgcaggtgtccaccagcaacatcagctcactggagggggcccggggcctcattgccgaggcggcgcagcttgggcccgtgggcggcgtcttcaacctggccgtggtcttgagagatggcttgctggagaaccagaccccagagttcttccaggacgtctgcaagcccaagtacagcggcaccctgaacctggacagggtgacccgagaggcgtgccctgagctggactactttgtggtcttctcctctgtgagctgcgggcgtggcaatgcgggacagagcaactacggctttgccaattccgccatggagcgtatctgtgagaaacgccggcacgaaggcctcccaggcctggccgtgcagtggggcgccatcggcgacgtgggcattttggtggagacgatgagcaccaacgacacgatcgtcagtggcacgctgccccagcgcatggcgtcctgcctggaggtgctggacctcttcctgaaccagccccacatggtcctgagcagctttgtgctggctgagaaggctgcggcctatagggacagggacagccagcgggacctggtggaggccgtggcacacatcctgggcatccgcgacttggctgctgtcaacctggacagctcactggcggacctgggcctggactcgctcatgagcgtggaggtgcgccagacgctggagcgtgagctcaacctggtgctgtccgtgcgcgaggtgcggcaactcacgctccggaaactgcaggagctgtcctcaaaggcggatgaggccagcgagctggcatgccccacgcccaaggaggatggtctggcccagcagcagactcagctgaacctgcgctccctgctggtgaacccggagggccccaccctgatgcggctcaactccgtgcagagctcggagcggcccctgttcctggtgcacccaatcgagggctccaccaccgtgttccacagcctggcctcccggctcagcatccccacctatggcctgcagtgcacccgagctgcgccccttgacagcatccacagcctggctgcctactacatcgactgcatcaggcaggtgcagcccgagggcccctaccgcgtggccggctactcctacggggcctgcgtggcctttgaaatgtgctcccagctgcaggcccagcagagcccagcccccacccacaacagcctcttcctgttcgacggctcgcccacctacgtactggcctacacccagagctaccgggcaaagctgaccccaggctgtgaggctgaggctgagacggaggccatatgcttcttcgtgcagcagttcacggacatggagcacaacagggtgctggaggcgctgctgccgctgaagggcctagaggagcgtgtggcagccgccgtggacctgatcatcaagagccaccagggcctggaccgccaggagctgagctttgcggcccggtccttctactacaagctgcgtgccgctgagcagtacacacccaaggccaagtaccatggcaacgtgatgctactgcgcgccaagacgggtggcgcctacggcgaggacctgggcgcggactacaacctctcccaggtatgcgacgggaaagtatccgtccacgtcatcgagggtgaccaccgcacgctgctggagggcagcggcctggagtccatcatcagcatcatccacagctccctggctgagccacgcgtgagcgtgcgggagggctaggcccgtgcccccgcctgccaccggaggtcactccaccatccccaccccaccccaccccacccccgccatgcaacgggattgaagggtcctgccggtgggaccctgtccggcccagtgccactgccccccgaggctgctagacgtaggtgttaggcatgtcccacccacccgccgcctcccacggcacctcggggacaccagagctgccgacttggagactcctggtctgtgaagagccggtggtgcccgtgcccgcaggaactgggctgggcctcgtgcgcccgtggggtctgcgcttggtctttctgtgcttggatttgcatatttattgcattgctggtagagacccccaggcctgtccaccctgccaagactcctcaggcagcgtgtgggtcccgcactctgcccccatttccccgatgtcccctgcgggcgcgggcagccacccaagcctgctggctgcggccccctctcggccaggcattggctcagcccgctgagtggggggtcgtgggccagtccccgaggagctgggcccctgcacaggcacacagggcccggccacacccagcggccccccgcacagccacccgtggggtgctgcccttatgcccggcgccgggcaccaactccatgtttggtgtttgtctgtgtttgtttttcaagaaatgattcaaattgctgcttggattttgaaatttactgtaactgtcagtgtacacgtctggaccccgtttcatttttacaccaatttggtaaaaatgctgctctcagcctcccacaattaaaccgcatgtgatctcc

SEQ ID NO: 24 - DNA, 5175 bp, Homo sapiens
  Homo sapiens low density lipoprotein receptor (familial hypercholesterolemia) (LDLR) (1)..(5175): representative cDNA sequence (NM_000527.2) as of 24 March 2003) for gene under Unigene ID Hs.213289
gccccgagtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgcccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctgtgaaggataagagaaacaggcccggggggaccaggatgacacctccatttctctccaggaagttttgagtttctctccaccgtgacacaatcctcaaacatggaagatgaaagggcaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcaccggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggcctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggtgggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaacaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtatggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaatg

SEQ ID NO: 25 - DNA, 1528 bp, Homo sapiens
  Homo sapiens nuclear receptor subfamily 1, group H, member 3 (NR1H3) (1)..(1528): representative cDNA sequence (NM_005693.1) as of 24 March 2003) for gene under Unigene ID Hs.347353
cagtgccttggtaatgaccagggctccagaaagagatgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccaggcgttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatgactgttctgtccccatattttctgttttcttggccggatggctgaggcctggtggctgcctcctagaagtggaacagactgagaagggcaaacattcctgggagctgggcaaggagatcctcccgtggcattaaaagagagtcaaagggt

SEQ ID NO: 26 - DNA, 3308 bp, Homo sapiens
  Homo sapiens mRNA for very low density lipoprotein receptor (1)..(3308): representative cDNA sequence (D16493.1) as of 24 March 2003)  for gene under Unigene ID Hs.73729
tttcggaagggctggtaacttgttgtgcggagcgaacggcggcggcggcggcaccatccaggcgggcaccatgggcacgtccgcgctctgggcgctctggctgctgctcgcgctgtgctgggcgccccgggagagcggcgccaccggaaccgggagaaaagccaaatgtgaaccctcccaattccagtgcacaaatggtcgctgtattacgctgttgtggaaatgtgatggggatgaagactgtgttgacggcagtgatgaaaagaactgtgtaaagaagacgtgtgctgaatctgacttcgtgtgcaacaatggccagtgtgttcccagccgatggaagtgtgatggagatcctgactgcgaagatggttcagatgaaagcccagaacagtgccatatgagaacatgccgcatacatgaaatcagctgtggcgcccattctactcagtgtatcccagtgtcctggagatgtgatggtgaaaatgattgtgacagtggagaagatgaagaaaactgtggcaatataacatgtagtcccgacgagttcacctgctccagtggccgctgcatctccaggaactttgtatgcaatggccaggatgactgcagcgatggcagtgatgagctggactgtgccccgccaacctgtggcgcccatgagttccagtgcagcacctcctcctgcatccccatcagctgggtatgcgacgatgatgcagactgctccgaccaatctgatgagtccctggagcagtgtggccgtcagccagtcatacacaccaagtgtccagccagcgaaatccagtgcggctctggcgagtgcatccataagaagtggcgatgtgatggggaccctgactgcaaggatggcagtgatgaggtcaactgtccctctcgaacttgccgacctgaccaatttgaatgtgaggatggcagctgcatccatggcagcaggcagtgtaatggtatccgagactgtgtcgatggttccgatgaagtcaactgcaaaaatgtcaatcagtgcttgggccctggaaaattcaagtgcagaagtggagaatgcatagatatcagcaaagtatgtaaccaggagcaggactgcagggactggagtgatgagcccctgaaagagtgtcatataaacgaatgcttggtaaataatggtggatgttctcatatctgcaaagacctagttataggctacgagtgtgactgtgcagctgggtttgaactgatagataggaaaacctgtggagatattgatgaatgccaaaatccaggaatctgcagtcaaatttgtatcaacttaaaaggcggttacaagtgtgaatgtagtcgtggctatcaaatggatcttgctactggcgtgtgcaaggcagtaggcaaagagccaagtctgatcttcactaatcgaagagacatcaggaagattggcttagagaggaaagaatatatccaactagttgaacagctaagaaacactgtggctctcgatgctgacattgctgcccagaaactattctgggccgatctaagccaaaaggctatcttcagtgcctcaattgatgacaaggttggtagacatgttaaaatgatcgacaatgtctataatcctgcagccattgctgttgattgggtgtacaagaccatctactggactgatgcggcttctaagactatttcagtagctaccctagatggaaccaagaggaagttcctgtttaactctgacttgcgagagcctgcctccatagctgtggacccactgtctggctttgtttactggtcagactggggtgaaccagctaaaatagaaaaagcaggaatgaatggattcgatagacgtccactggtgacagcggatatccagtggcctaacggaattacacttgaccttataaaaagtcgcctctattggcttgattctaagttgcacatgttatccagcgtggacttgaatggccaagatcgtaggatagtactaaagtctctggagttcctagctcatcctcttgcactaacaatatttgaggatcgtgtctactggatagatggggaaaatgaagcagtctatggtgccaataaattcactggatcagagctagccactctagtcaacaacctgaatgatgcccaagacatcattgtctatcatgaacttgtacagccatcaggtaaaaattggtgtgaagaagacatggagaatggaggatgtgaatacctatgcctgccagcaccacagattaatgatcactctccaaaatatacctgttcctgtcccagtgggtacaatgtagaggaaaatggccgagactgtcaaagtactgcaactactgtgacttacagtgagacaaaagatacgaacacaacagaaatttcagcaactagtggactagttcctggagggatcaatgtgaccacagcagtatcagaggtcagtgttcccccaaaagggacttctgccgcatgggccattcttcctctcttgctcttagtgatggcagcagtaggtggctacttgatgtggcggaattggcaacacaagaacatgaaaagcatgaactttgacaatcctgtgtacttgaaaaccactgaagaggacctctccatagacattggtagacacagtgcttctgttggacacacgtacccagcaatatcagttgtaagcacagatgatgatctagcttgacttctgtgacaaatgttgacctttgaggtctaaacaaataatacccccgtcggaatggaaccgagccagcagctgaagtctctttttcttcctctcggctggaagaacatcaagatacctttgcgtggatcaagcttgtgtacttgaccgtttttatattacttttgtaaatattcttgtccacattctacttcagctttggatgtggttaccgagtatctgtaacccttgaatttctagacagtattgccacctctggccaaatatgcactttccctagaaagccatattccagcagtgaaacttgtgctatagtgtataccacctgtacatacattgtataggccatctgtaaatatcccagagaacaatcactattcttaagcactttgaaaatatttctatgtaaattattgtaaactttttcaatggttgggacaatggcaataggacaaaacgggttactaagatgaaattgccaaaaaaatttataaactaattttgtacgtatgaatgatatctttgacctcaatggaggtttgcaaagactgagtgttcaaactactgtacattttttttcaagtgctaaaaaattaaaccaagcagcttaaccatg

SEQ ID NO: 27 - DNA, 18 bp, Homo sapiens
  ABCA1 forward primer (1)..(18)
aacccaccacaggcatgg

SEQ ID NO: 28 - DNA, 22 bp, Homo sapiens
  ABCA1 reverse primer (1)..(22)
acacttagggcacaattccaca

SEQ ID NO: 29 - DNA, 19 bp, Homo sapiens
  ABCA1 probe (1)..(19)
tcccaaagcccggcggttc

SEQ ID NO: 30 - DNA, 21 bp, Homo sapiens
  ABCG1 forward primer (1)..(21)
ccctccagtcatgttcttcga

SEQ ID NO: 31 - DNA, 18 bp, Homo sapiens
  ABCG1 reverse primer (1)..(18)
atgatggagcgaccccct

SEQ ID NO: 32 - DNA, 21 bp, Homo sapiens
  ABCG1 probe (1)..(21)
ccctccagtcatgttcttcga

SEQ ID NO: 33 - DNA, 20 bp, Homo sapiens
  APOC1 forward primer (1)..(20)
catgaggctcttcctgtcgc

SEQ ID NO: 34 - DNA, 19 bp, Homo sapiens
  APOC1 reverse primer (1)..(19)
tgggccttccaagacgatc

SEQ ID NO: 35 - DNA, 23 bp, Homo sapiens
  APOC1 probe (1)..(23)
cccggtcctggtggtggttctgt

SEQ ID NO: 36 - DNA, 20 bp, Homo sapiens
  APOE forward primer (1)..(20)
cgttgctggtcacattcctg

SEQ ID NO: 37 - DNA, 19 bp, Homo sapiens
  APOE reverse primer (1)..(19)
gctctgtctccaccgcttg

SEQ ID NO: 38 - DNA, 22 bp, Homo sapiens
  APOE probe (1)..(22)
caggatgccaggccaaggtgga

SEQ ID NO: 39 - DNA, 25 bp, Homo sapiens
  ASM3A forward primer (1)..(25)
gaatctaaagggagagtccatctgg

SEQ ID NO: 40 - DNA, 20 bp, Homo sapiens
  ASM3A reverse primer (1)..(20)
tccggctgcaaatcttcaat

SEQ ID NO: 41 - DNA, 31 bp, Homo sapiens
  ASM3A probe (1)..(31)
agctggagtatatcctgacccagacctacga

SEQ ID NO: 42 - DNA, 19 bp, Homo sapiens
  CDC42EP forward primer (1)..(19)
tcgggtatgagcccctgag

SEQ ID NO: 43 - DNA, 19 bp, Homo sapiens
  CDC42EP reverse primer (1)..(19)
ggaggtgggtcaggctgtt

SEQ ID NO: 44 - DNA, 28 bp, Homo sapiens
  CDC42EP probe (1)..(28)
ttgactgccggttatttttctgtcctgg

SEQ ID NO: 45 - DNA, 19 bp, Homo sapiens
  CXCR4 forward primer (1)..(19)
gcaggacctgtggccaagt

SEQ ID NO: 46 - DNA, 21 bp, Homo sapiens
  CXCR4 reverse primer (1)..(21)
cgctctggaatgttcagttcc

SEQ ID NO: 47 - DNA, 37 bp, Homo sapiens
  CXCR4 probe (1)..(37)
ttagttgctgtatgtctcgtggtaggactgtagaaaa

SEQ ID NO: 48 - DNA, 21 bp, Homo sapiens
  FASN forward primer (1)..(21)
ttgcattgctggtagagaccc

SEQ ID NO: 49 - DNA, 19 bp, Homo sapiens
  FASN reverse primer (1)..(19)
cacacgctgcctgaggagt

SEQ ID NO: 50 - DNA, 21 bp, Homo sapiens
  FASN probe (1)..(21)
caggcctgtccaccctgccaa

SEQ ID NO: 51 - DNA, 20 bp, Homo sapiens
  LDLR forward primer (1)..(20)
ccccagggacaaaacactgt

SEQ ID NO: 52 - DNA, 20 bp, Homo sapiens
  LDLR reverse primer (1)..(20)
gctccgaaaccagaaaggct

SEQ ID NO: 53 - DNA, 20 bp, Homo sapiens
  LDLR probe (1)..(20)
ccccccagtgcagggaaccg

SEQ ID NO: 54 - DNA, 19 bp, Homo sapiens
  NR1H3 forward primer (1)..(13)
tgtaaccggcgctcctttt

SEQ ID NO: 55 - DNA, 15 bp, Homo sapiens
  NR1H3 reverse primer (1)..(15)
tggtgccatgggcca

SEQ ID NO: 56 - DNA, 21 bp, Homo sapiens
  NR1H3 probe (1)..(21)
tgaccggcttcgagtcacgcc

SEQ ID NO: 57 - DNA, 20 bp, Homo sapiens
  VLDLR forward primer (1)..(20)
caaataatacccccgtcgga

SEQ ID NO: 58 - DNA, 20 bp, Homo sapiens
  VLDLR reverse primer (1)..(20)
ccagccgagaggaagaaaaa

SEQ ID NO: 59 - DNA, 27 bp, Homo sapiens
  VLDLR probe (1)..(27)
tggtaaccgagccagcagctgaagtct